(19) United States
(12) Patent Application Publication
(10) Pub. No.: US 2024/0173364 A1
(43) Pub. Date: May 30, 2024

(54) METHODS, COMPOSITIONS, AND SYSTEMS FOR TREATING DENTAL CARIES AND A PERIODONTAL DISEASE BASED ON MICROBIOME MODULATION THROUGH ORAL MICROBIOME TRANSPLANT (OMT)

(71) Applicants: The Penn State Research Foundation, University Park, PA (US); The University of Adelaide, Adelaide (AU)

(72) Inventors: Peter Zilm, Adelaide (AU); Lisa Jamieson, Adelaide (AU); Laura Weyrich, University Park, PA (US)

(21) Appl. No.: 18/521,383

(22) Filed: Nov. 28, 2023

Related U.S. Application Data
(60) Provisional application No. 63/385,063, filed on Nov. 28, 2022.

(51) Int. Cl. A61K 35/742; C12M 1/12; C12N 1/20; C12R 1/07; C12R 1/21

(52) U.S. Cl. CPC A61K 35/742; C12M 23/06; C12M 25/14; C12N 1/205; C12N 2500/14; C12N 2500/32; C12N 2500/38; C12N 2500/46; C12N 2500/74; C12N 2500/84; C12N 2501/998; C12N 2513/00; C12N 2523/00; C12N 2533/18; C12R 2001/07; C12R 2001/21

(57) ABSTRACT

Provided herein are methods, compositions, and systems for treating dental caries and a periodontal disease based on microbiome modulation through oral microbiome transplant (OMT). The original oral microbial composition was obtained from the dental plaque of healthy donors, and then modulated by growing a biofilm in vitro in a growth medium. The modulated oral microbial composition has a different microbial diversity than the original oral microbial composition. In the modulated oral microbial compositions, the abundance of some bacterial species changes, some new bacterial species are present, and some original bacterial species are no longer present. The modulated oral microbial composition is transplanted to the recipient's oral cavities through OMT to treat dental caries or a periodontal disease.

### CROSS REFERENCE TO RELATED APPLICATIONS

This application claims priority to U.S. Provisional Application No. 63/385,063, filed Nov. 28, 2022, which is hereby incorporated by reference in its entirety.

### FIELD

Provided herein are methods, compositions, and systems for treating dental caries and a periodontal disease based on microbiome modulation through oral microbiome transplant (OMT).

### BACKGROUND

Dental caries is the most common noncommunicable disease worldwide, impacting nearly 3.5 billion people, and remains the most prevalent chronic disease in the United States. Globally, dental diseases directly cost $356.8 billion in a single year (2015), and an additional $187.6 billion in indirect costs. Yet, technology introduced in the 1950s is still employed to prevent oral disease (i.e., tooth brushing, flossing, mouth wash, and chewing gums) combined with routine visits to dental hygienists, accessed only by the most fortunate. New treatments for this disease are desperately needed. While Streptococcus mutans and other Streptococcus species can play key roles in caries development, continual lifestyle changes and a lack of understanding of the roles that additional species within the biofilm play in caries development have hindered our ability to effectively cure dental caries. These species are part of a vast network of microorganisms—bacteria, archaea, viruses, fungi, and protists—that live within the human body—the microbiome. In fact, the human body contains 1.3× more microorganisms than human cells, encompassing over 1,800 species of bacteria, fungi, parasites, and viruses (microbiota) that account for 1.4 kg of human body weight—equal in mass to the human brain. These microbes also contain >99% of the genetic diversity within the human body, as each human contains 2-5 million microbial genes compared to <30,000 human genes. Specifically in the mouth, alterations in oral microbiota (e.g., a dysbiosis or shift from a comparative, healthy community) are linked to nearly all oral diseases examined to date, including caries, periodontal disease, gingivitis, halitosis, and oral cancers.

Dental caries (cavities or dental decay) impact young children, adults, and the elderly alike. Dental caries affects over 78% of United States children, and more than 90% of children in other countries, such as Mexico. While a caries-associated plaque biofilm was traditionally thought to be modulated by a single acidogenic bacteria, Streptococcus mutans, it is now known that caries etiology is linked to several species within the oral microbiome—the hundreds of bacteria, archaea, viruses, fungi, and parasites living in the human mouth. Continual lifestyle changes and a key lack of understanding regarding synergistic activities with other oral microbes have hindered our ability to effectively cure this disease.

Conventional methods for both the treatment of dental caries and periodontal disease involve causative therapy. The treatment of dental caries involves the removal of demineralized tissue and replacing it with filling materials. The primary treatment for periodontal disease is mechanical removal of the plaque biofilm and management of the potential risk factors. Although the methods are effective in managing dental caries and periodontal disease, they fall short of completely preventing and arresting the diseases, resulting in potential short term benefits and the likely re-emergence of diseases. The failure rates of dental restorative materials are high, at an annual rate of 8%, due to secondary caries and bulk fracture. Earlier interventional options, such as the application of resin-based sealants on permanent molars, reduce caries by only 11%-51% for a limited time period of up to 48 months. In the case of periodontal disease, almost all on periodontal maintenance therapy continue to show signs of progressive clinical attachment loss, with one to two-thirds of people losing at least one tooth during an extended period of periodontal maintenance care. It is clearly imperative to find necessary ways for better preventing and managing periodontitis and dental caries. This is especially true considering higher rates of tooth loss in aging populations and severe rates of disease in young children. New preventative and treatment approaches leveraging oral microbiome research are needed to develop novel therapeutics that move dentistry beyond a reactive approach to treating dental caries. Oral microbiome therapy (OMT) may offer an effective alternative for the treatment of common oral diseases.

Oral microbiome transplant (OMT) therapy uses an ecological approach to preventing dental caries and can revolutionize the way that this disease is understood, treated, and managed. An OMT typically involves harvesting donor dental plaque or saliva from healthy donors and transplanting it into a recipient (i.e., someone with severe caries). The microbiota from an OMT super donor may have the following characteristics: (1) possesses limited abundances of ‘red’ complex microbes and known cariogenic species (e.g., Streptococcus mutans); (2) has no transmissible or infectious pathogens (herpes virus, human immunodeficiency virus, etc.); (3) successfully grow in in-vitro model, exhibiting higher proportion of live bacterial cells than dead bacterial cells; (4) be able to effectively grow in a host by causing a shift in the microbiome profile of an individual towards that of a donor; and (5) is efficacious against oral disease in a rodent model (e.g., reduces inflammation) with limited negative outcomes (e.g., oral infection or increases in oral disease markers).

While this has been trialled in a murine model of mucositis and canines with periodontal disease, OMTs have not yet been deployed in humans nor against dental caries, despite the fact that microbiota transplants are utilized in the gut with incredible success for some diseases (i.e., fecal microbiota transplants (FMTs) altered the success rates of treating recurrent C. difficile infections from ˜28% with antibiotics to over 85% with FMT 24-26). The OMT therapy can help individuals who are susceptible to severe caries, have carious lesions that are recalcitrant to treatment, or live in situations with limited access to routine dental care.

Dental caries are characterized by the demineralization of a tooth's enamel by microbes within dental biofilms adherent to the tooth surface. In caries, the plaque biofilm is thought to be modulated by acidogenic bacteria, such as Streptococcus mutans. S. mutans is well adapted to tolerate an acidic environment and, in high numbers, is considered to be an opportunistic pathogen due to its ability to liberate lactic acid and proliferate in acidic environments. Caries remains one of the most preventable chronic diseases, especially in disadvantaged populations. A reactive approach to dental decay in children has only continued to result in an increase in childhood caries since the late 1990s, with half of all children having decay in at least one permanent tooth by the age of twelve. Novel solutions that prevent caries before they start are desperately needed.

The OMT treatment could be a lifelong or prolonged solution to shifting oral microbiota and perhaps limiting caries development throughout life. An application of OMT therapy at an effective stage of life (i.e., during the eruption of a permanent dentition) may provide a way to positively change a person's oral microbiota for years afterward. In addition, the aging population also shows increased dental caries due to poor care and management in aged care facilities. An OMT application associated with the introduction of dental prosthetics (e.g., dentures) may be another key life stage to alter disease-associated microbiota.

OMTs could be employed to aid in the treatment of numerous other oral diseases, including periodontal disease, cancer, mucositis, halitosis, and dry mouth. Further, periodontal disease is also a risk factor for developing several systemic diseases—many of which are listed in the top ten noncommunicable diseases—including diabetes, cardiovascular disease, chronic kidney disease, osteoporosis, and Alzheimer's disease. OMT therapy may be able to directly address oral health issues and contribute measurable improvements to other non-infectious diseases.

### SUMMARY

Provided herein is a method for treating dental caries or periodontitis in a subject in need of, the method comprising:
a) collecting dental plaque from a donor;
b) growing a biofilm in vitro in a growth medium to modulate the oral microbial composition of the collected dental plaque from step a); and
c) transplanting the biofilm into an oral cavity of a recipient.

Also provided herein is a method for modulating the oral microbial composition of a recipient, the method comprising:
a) collecting dental plaque from a donor;
b) growing a biofilm in vitro in a growth medium to modulate the oral microbial composition of the collected dental plaque from step a); and
c) transplanting the biofilm into an oral cavity of a recipient.

Further provided herein is modulated oral microbial composition prepared by:
a) collecting dental plaque from a donor;
b) growing a biofilm in vitro in a growth medium to modulate the microbial composition of the collected dental plaque from step a).

Further provided herein is a system comprising a flow cell and at least one hydroxyapatite (HA) disc for use to modulate an oral microbial composition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 (top) shows the flow cell system setup that consists of six major components: a medium reservoir (A), a peristaltic pump (B), a break-line (C), the flow cell (D), a slide heating tray (E) and a spent medium container (F). FIG. 1 (bottom) shows 16 different flow cells in parallel.

FIG. 2 shows the general protocol for modulating oral microbial composition through OMT.

FIG. 3 shows SEM (top) and CLSM (bottom) used to visualize cell morphology and living cells of 14-day-old in vitro biofilms from two subjects. (Top) groups of oral streptococci and several different microbial morphologies, representing a diverse mixture of microbes, can be observed. (Bottom) live:dead staining was used to examine if cells 14 days post inoculation were alive; on average, 72% of the cells living in the biofilm on day 14 were alive.

FIG. 4 shows the protocol for OMT in a rat model.

FIG. 5 shows an intragroup comparison of mean caries scores between cages for each of the four groups. A. Group 1—control cariogenic diet and control CMC gel (carboxymethylcellulose gel that comprises donor microbiota in hydrogel). B. Group 2—Cariogenic diet and Streptococcus mutans in CMC gel. C. Group 3—Cariogenic diet and oral microbiome transplant (OMT). Group 4—Cariogenic diet, Streptococcus mutans, and OMT in CMC gel. Paired t-test was performed, and there was no significant difference between the groups. For groups 3 and 4, 3A/4A is donor 1, and 3B/4B is donor 2. FIGS. 5-7 use a caries scoring system on a scale of 1-5.

FIG. 6A shows that ANOVA was performed for multiple group comparisons. There was a significant difference between the groups. FIG. 6B shows a post hoc Tukey test for comparison among multiple groups. A decrease in the mean caries score trend was observed. A significant difference was observed when Group 1 CMC group gel was compared with Group 4 OMT+Streptococcus mutans.

FIG. 7 shows the total number of healthy sites (no caries) and affected with caries severity for each group. The first group had no caries or healthy sites for each treatment group. The second group is mild caries, sites with only enamel involvement. The third group is moderate caries, having cavitation with dentin involvement. The fourth group is severe caries involvement, having more than one-third of dentin involved. The results show that OMT+S. mutans have no severe carious affected teeth.

FIG. 8 shows an intragroup comparison of mean caries scores between cages for each of the four groups. A. Group 1—control cariogenic diet and control gel CMC. B. Group 2—Cariogenic diet and Streptococcus mutans in CMC gel. C. Group 3—Cariogenic diet and oral microbiome transplant (OMT). Group 4—Cariogenic diet, Streptococcus mutans, and OMT in CMC gel. For groups 3 and 4, 3A/4A is donor 1, and 3B/4B is donor 2. FIGS. 8-10 use a binary caries scoring system (either having caries (1) or no caries (0)).

FIG. 9A shows that ANOVA was performed for multiple group comparisons. There was a significant difference between the four groups. FIG. 9B shows a post hoc Tukey test for comparison among multiple groups. A decrease in the mean caries score trend was observed. A significant difference was observed between Group 1 CMC vs. Group 3 Oral microbiome transplant only; Group 1 CMC group vs. Group 4 OMT+Streptococcus mutans; and Group 2. S. mutans vs. Group 3 OMT.

FIG. 10 shows the total number of healthy sites (no caries) and affected with caries severity for each group. The first group had no caries or healthy sites for each treatment group. The second group has caries, showing cavitation with enamel and dentin involvement. The results show that Group 3 OMT and Group 4 OMT+S. mutans have fewer carious affected teeth.

FIG. 11 shows a supragingival dental plaque biofilm formation on HA discs following 14 days of growth using ASM or SHI medium. A) SEM images of HA discs grown in ASM from all 6 volunteers, 1-6. B) SEM images of HA discs grown in SHI from 3 volunteers 4-6. Images were taken from three random regions of each HA disc using the magnifications: 1,000×, 5,000× and 20,000×. Scale bars=2 μm, 10 μm and 50 μm, respectively.

FIG. 12 shows cell viability and 16S rRNA sequencing analysis on microbiota grown in ASM for 14-days. A) Percentage viability of supragingival biofilms established in the established biofilm grown using either ASM or SHI medium. 73.5% and 72.4% of cells in biofilms were alive, and 26.5% and 27.6% were dead in the ASM and SHI mediums, respectively. Error bars represent mean±S.D. ****p<0.0001, ns: non-significant. B) PCoA plot indicating differentiation of microbiota. Axis 1 explains 31.04% of the variation in the dataset; axis 2 explains 12.00% of the variation and axis 3 explains 7.098% of the variation. The bacterial samples including inoculated and collected plaque, T14 biofilms and planktonic cells, cluster more distantly along the first ordination axis whereas control samples representing the sample blanks and negative controls cluster closely together. C) Comparison of β-diversity of the collected, inoculated and T14 biofilms grown in ASM. Axis 1 explains 51.75% of the variation in the dataset; axis 2 explains 21.36% of the variation and axis 3 explains 11.65% of the variation. D) Change in the dominant phyla from T0 to T14.

FIG. 13 shows a Linear Discriminant Analysis (LDA) Effect Size (LEfSe) analysis at the species level to compare the oral microbiome profiles of the donors between inoculated plaque (T0) and T14 biofilms grown in ASM.

FIG. 14 shows a 16S rRNA sequencing analysis on microbiota grown in SHI. A) PCoA plot indicating differentiation of microbiota. Axis 1 explains 23.38% of the variation in the dataset; axis 2 explains 12.40% of the variation and axis 3 explains 8.776% of the variation. Inoculated plaque, biofilms grown in FCs, planktonic cells and spent medium overlap closely with medium, PBS, FC control and EBCs on the first ordination axis. B) Comparison of 0-diversity of the inoculated and T14 biofilms grown in SHI. Axis 1 explains 34.77% of the variation in the dataset; axis 2 explains 27.46% of the variation and axis 3 explains 11.83% of the variation. The inoculated plaque samples (with the exception of one inoculated sample) cluster closely together along the second ordination axis with T14 biofilms.

### DETAILED DESCRIPTION

Unless defined otherwise, all technical and scientific terms have the same meaning as is commonly understood by one of ordinary skill in the art to which the embodiments disclosed belong.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “biofilm” means a layer of bacteria that can accumulate inside or on the substrate of a living subject.

As used herein, the term “cancer” means a spectrum of pathological symptoms associated with the initiation or progression, as well as metastasis, of malignant tumors.

As used herein, the term, “compound” means all stereoisomers, tautomers, and isotopes of the compounds described herein.

As used herein, the terms “comprising” (and any form of comprising, such as “comprise,” “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together two elements in an in vitro system or an in vivo system.

As used herein, the term “dental plaque” means a biofilm of microorganisms that grows on surfaces within the mouth.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the term “inoculate” means introducing.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from X to Y” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “microbiome” or “microbiota” means a collection of all microbes, such as bacteria, fungi, and viruses. In some embodiments, “microbial species” and “bacterial species” are used interchangeably. In some embodiments, “microbiome” and “microbiota” are used interchangeably.

As used herein, the term “microbial composition” means a composition comprising all microbes. As used herein, the term “oral microbial composition” means a composition comprising all oral microbes of a sample. In some embodiments, the sample is a dental plaque.

As used herein, the term “microbial diversity” describes the number of each different species of microbes present and their distribution.

As used herein, the term “modulate” means modifying, changing, or varying. The term “modulate the oral microbial composition” means modifying, changing, or varying the identity of at least one microbial species or the amount of at least one microbial species.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of the progression of the disorder, or improved treatment, healing, elimination or amelioration of a disorder, or side effects. The amount needed to elicit the therapeutic response can be determined based on the age, health, size, and sex of the subject. Optimal amounts can also be determined based on monitoring of the subject's response to treatment.

As used herein, the term “oral microbiome transplantation” or “OMT” means introducing health-associated oral microbiota into the oral cavity of a diseased patient or a healthy patient for future disease prevention.

As used herein, the term “periodontal disease” means a disease that is the result of infections and inflammation of the gums and bones that surround and support the teeth. In some embodiments, the term “periodontal disease” means gum disease. In some embodiments, the term “periodontal disease” includes gingivitis, necrotizing periodontal disease, and peritonitis (including aggressive peritonitis, chronic peritonitis, and systemic peritonitis).

As used herein, the term “SHI medium” means an enriched growth medium capable of supporting in vitro biofilms with similar diversity to healthy supragingival inocula.

As used herein, the terms “treat,” “treated,” or “treating” means therapeutic treatment wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment.

At various places in the present specification, substituents of compounds may be disclosed in groups or in ranges. It is specifically intended that embodiments include each and every individual sub-combination of the members of such groups and ranges. For example, the term “C₁₋₆alkyl” is specifically intended to individually disclose methyl, ethyl, propyl, C₄alkyl, C₅alkyl, and C₆alkyl.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

In some embodiments/in one embodiment/in another embodiment/in an additional embodiment, the composition is formulated as a capsule, a pill, a cachet, a tablet, a granule, multi-particulates, mini-tablets, or powder.

Provided herein is a method for treating dental caries or a periodontal disease in a subject in need of, the method comprising:
a) collecting dental plaque from a donor;
b) growing a biofilm in vitro in a growth medium to modulate the oral microbial composition of the collected dental plaque from step a); and
c) transplanting the biofilm into the oral cavity of a recipient.

In some embodiments, the growth medium is an artificial saliva (ASM) or SHI medium.

The artificial saliva (ASM) or SHI medium can be modified ASM or SHI.

In some embodiments, the artificial saliva (ASM) comprises about 0.50 g/L tryptone, about 0.50 g/L neutralized bacteriological peptone, about 0.625 g/L type III porcine gastric mucin, about 0.25 g/L yeast extract, about 0.05 g/L KCl, about 0.05 g/L CaCl₂), about 0.088 g/L NaCl, and A mg/L haemin.

In some embodiments, the SHI medium has supplements. In some embodiments, SHI medium comprises about 5.0 g/L tryptone, about 10.0 g/L peptone, about 2.5 g/L type III porcine mucin, about 5.0 g/L yeast extract, about 1.0 mg/L Vitamin K (Sigma-Aldrich), about 2.5 g/L KCl, about 5.0 mg/L haemin, about 0.174 g/L L-arginine, about 0.06 g/L urea, about 5% v/v sheep blood, and about 10.0 mg/L N-acetylmuramic acid.

In some embodiments, the method further comprises step a-1) prior to step a):
a-1) providing a flow cell and at least one hydroxyapatite (HA) disc.

The previous model includes the systems that also grow dental plaque in individual wells, similar to how human cell lines are grown or cultured, which does not mimic the conditions of the human mouth. In contrast, the present application provides methods for growing dental biofilms in a 3D-printed flow cell, which mimics the salivary flow and environment of the human mouth. This is a critical point, as the flow cell is engineered to move ASM medium (that directly mimics human saliva) at rates and in dispersion patterns (similar to the shape of a V or how geese fly) that mimic how saliva spreads in the mouth around teeth. In the present application, the hydroxyapatite discs were placed (which is the material that human tooth enamel is comprised) in the bottom of the flow cell, which allows dental or oral biofilms to grow on hard tissues (as they would on a tooth) to form and grow naturally on enamel. This system is also portable and expandable, allowing the growth of dental plaque in different locations and in different amounts (for example, up to 64 discs). Surprisingly and advantageously, this system is able to propagate up to 230 different species. Putative oral pathogens, such as Porphyromonas gingivalis, Treponema denticola, and Tannerella forsythia were removed from the flow cell, as well as human associated viruses that require human tissue to propagate (such as HIV and herpes).

In some embodiments, the method further comprises step a-2) after step a-1):
a-2) sterilizing the flow cell.

In some embodiments, the method further comprises step a-3) after step a-2):
a-3) filling the flow cell with the growth medium.

In some embodiments, the growth medium is devoid of sucrose for about 24 hours to allow pellicle formation.

In some embodiments, the method further comprises step a-4) prior to step a):
a-4) mixing the dental plaque with a phosphate buffer solution (PBS) to form a first mixture.

In some embodiment, step a-4) comprises homogenizing dental plaque in 200 uL of sterile phosphate saline buffer by vortexing. The homogenization step may be followed by assessing pH using a glucose challenge test.

In some embodiments, the method further comprises step a-5) after step a-4):
a-5) adding the mixture to the growth medium to form a second mixture.

In some embodiments, step a-5) comprises mixing 100 uL of homogenized dental plaque with 1 mL of 25 modified artificial salivary medium (ASM) to create innoculate. The innoculate may be the second mixture.

In some embodiments, the method further comprises step a-6) after step a-5):
a-6) inoculating the second mixture into the flow cell.

In some embodiments, step a-6) comprises seeding the sterilized flow cell system using 100 uL of inoculate through the input value.

In some embodiments, the method further comprises step a-7) after step a-6):
a-7) flowing the flow cell with the growth medium.

In some embodiments, the method further comprises step a-8) after step a-7):
a-8) growing the biofilm on the HA disc.

Step a-8) may comprise mixing the flow cell once each day and ensuring that liquid is flowing through the system. The growth may occur for at least 10 days. In some embodiments, the growth occurs for about 14 days. The growth may occur at about 36° C., or the growth may occur at about 37° C. At the end of the growth, the method may comprise opening the flow cell, removing at least two HA discs, verifying growth of live microorganisms using live: dead microscopy, and performing 16S ribosomal RNA sequencing to define the species that were maintained and grown in dental plaque.

In some embodiments, at least 60% of the cells in the biofilm are viable. In some embodiments, at least 65% of the cells in the biofilm are viable. In some embodiments, at least 70% of the cells in the biofilm are viable.

In some embodiments, the biofilm comprises at least 200 bacterial species. In some embodiments, the biofilm comprises at least 220 bacterial species. In some embodiments, the biofilm comprises at least 240 bacterial species. In some embodiments, the biofilm has about 252 bacterial species.

In some embodiments, the microbial composition collected from the dental plaque comprises more than 280 species. In some embodiments, the microbial composition collected from the dental plaque comprises more than 300 species. In some embodiments, the microbial composition collected from the dental plaque comprises about 312 species.

In some embodiments, the microbial diversity in the biofilm is reduced relative to the microbial diversity in the collected dental plaque.

In some embodiments, the bacterial diversity in the biofilm is reduced relative to the bacterial diversity in the collected dental plaque.

In some embodiments, the number of bacterial species in the biofilm is less than the number of bacterial species in the collected dental plaque.

In some embodiments, the abundance of at least one bacterial species in the biofilm is less than the abundance of the at least one bacterial species in the collected dental plaque.

In some embodiments, at least one bacterial species that existed in the collected dental plaque no longer exists in the biofilm.

In some embodiments, the diversity of the microbial composition in the biofilm is different from the diversity of the microbial composition collected from the dental plaque. The diversity refers to the number of bacterial species present in the composition. In some embodiments, the biofilm reduces the diversity of the bacterial species by at least 10% relative to the collected dental plaque. In some embodiments, the biofilm reduces the diversity of the bacterial species by at least 15% relative to the collected dental plaque. In some embodiments, the biofilm reduces the diversity of the bacterial species by at least 20% relative to the collected dental plaque. In some embodiments, the biofilm reduces the diversity of the bacterial species by at least 20% relative to the collected dental plaque. In some embodiments, the biofilm reduces the diversity of the bacterial species by about 10% to about 20% relative to the collected dental plaque. In some embodiments, the biofilm reduces the diversity of the bacterial species by about 10% to about 22% relative to the collected dental plaque. In some embodiments, the biofilm reduces the diversity of the bacterial species by about 10% to about 40% relative to the collected dental plaque.

In some embodiments, the biofilm comprises aerobic bacteria.

In some embodiments, the biofilm comprises aerobic and/or anaerobic bacteria.

In some embodiments, the biofilm comprises aerobic and anaerobic bacteria (such as Tannerella forsythia, Porphyromonas gingivalis, and a combination thereof).

In some embodiments, the biofilm comprises aerobic and anaerobic bacteria. In some embodiments, the diversity of anaerobic bacteria in the biofilm is reduced relative to the diversity of anaerobic bacteria in the collected dental plaque. In some embodiments, the number of anaerobic bacterial species in the biofilm is less than the number of anaerobic bacterial species in the collected dental plaque. In some embodiments, the abundance of at least one anaerobic bacterial species in the biofilm is less than the abundance of the at least one anaerobic bacterial species in the collected dental plaque. In some embodiments, at least one anaerobic bacterial species that existed in the collected dental plaque no longer exists in the biofilm. In some embodiments, the anaerobic bacteria comprise Tannerella forsythia, Porphyromonas gingivalis, and a combination thereof.

In some embodiments, the microbiome of the biofilm comprises at least one bacterial species that does not exist in saliva or in the dental plaque.

In some embodiments, the microbiome of the biofilm has reduced the abundance of at least one bacterial species.

In some embodiments, the microbiome of the biofilm has reduced abundance in Actinobacteria, Fusobacteria, or a combination thereof relative to the microbiome of the collected dental plaque.

In some embodiments, the microbiome of the biofilm has reduced abundance in Actinobacteria, Fusobacteria, or a combination thereof relative to the microbiome of the collected dental plaque wherein the growth medium is ASM.

In some embodiments, the microbiome of the biofilm has increased abundance in Bacteriodetes, Firmicutes, and a combination thereof relative to the microbiome of the collected dental plaque.

In some embodiments, the microbiome of the biofilm has increased abundance in Bacteriodetes, Firmicutes, and a combination thereof relative to the microbiome of the collected dental plaque wherein the growth medium is ASM.

In some embodiments, the microbiome of the biofilm has increased abundance in Bacilli spp., Haemophilus parainfluenzae, or a combination thereof relative to the microbiome of the collected dental plaque.

In some embodiments, the microbiome of the biofilm has increased abundance in Bacilli spp., Haemophilus parainfluenzae, or a combination thereof relative to the microbiome of the collected dental plaque wherein the growth medium is ASM.

In some embodiments, the microbiome of the biofilm has reduced abundance or removed the abundance of oral pathogens. The oral pathogen may require human tissue to grow. In some embodiments, the oral pathogen comprises Porphyromonas gingivalis, Treponema denticola, Tannerella forsythia, or any of the combinations thereof.

In some embodiments, the biofilm reduces the proliferation of acidogenic and aciduric species or the proliferation of obligate anaerobic species.

In some embodiments, the biofilm reduces the proliferation of Streptococcus mutans.

In some embodiments, the thickness of the biofilm is in the range of about 25 μm to about 55 μm. In some embodiments, the thickness of the biofilm is in the range of about 15 μm to about 65 μm. In some embodiments, the thickness of the biofilm is in the range of about 5 μm to about 75 μm. In some embodiments, the thickness of the biofilm is in the range of about 1 μm to about 100 μm. In some embodiments, the thickness of the biofilm is in the range of about 0.1 μm to about 1000 μm.

In some embodiments, the biovolume of the biofilm is in the range of about 4 μm³ to about 8 μm³. In some embodiments, the biovolume of the biofilm is in the range of about 1 μm³ to about 20 μm³. In some embodiments, the biovolume of the biofilm is in the range of about 0.1 μm³ to about 100 μm³.

Provided herein is also a method for modulating the oral microbial composition that incorporates all the embodiments above.

Provided herein is also a method for modulating the oral microbial composition of a recipient that incorporates all the embodiments above.

Provided herein is also a method for other microbiota-related disorders or diseases. The method incorporates all the embodiments above.

Provided herein is also a method for treating cancer, mucositis, halitosis, or dry mouth. The method incorporates all the embodiments above.

Provided herein is further a modulated oral microbial composition prepared by:
a) collecting dental plaque from a donor; and
b) growing a biofilm in vitro in a growth medium to modulate the microbial composition of the collected dental plaque from step a).

The composition is prepared using the methods described above, incorporating all the embodiments above.

Provided herein is further a system comprising a flow cell and at least one hydroxyapatite (HA) disc for use to modulate an oral microbial composition.

The flow cell may be 3D printed. The flow cell may mimic the salivary flow and environment of the human mouth. The flow cell may be configured to move the growth medium at rates and in dispersion patterns that mimic how saliva spreads in the mouth around teeth.

The HA disc may be placed at the bottom of the flow cell. The HA disc may be configured to allow a dental or oral biofilm to grow.

In some examples, disclosed methods, systems, and compositions involve one or more of the following clauses:
1. A method for treating dental caries or a periodontal disease in a subject in need of, the method comprising:
a) collecting dental plaque from a donor;
b) growing a biofilm in vitro in a growth medium to modulate the oral microbial composition of the collected dental plaque from step a); and
c) transplanting the biofilm into the oral cavity of a recipient.
2. The method of clause 1, wherein the growth medium is an artificial saliva (ASM) or SHI medium.
3. The method of clause 2, wherein the artificial saliva (ASM) comprises about 0.50 g/L tryptone, about 0.50 g/L neutralized bacteriological peptone, about 0.625 g/L type III porcine gastric mucin, about 0.25 g/L yeast extract, about 0.05 g/L KCl, about 0.05 g/L CaCl₂), about 0.088 g/L NaCl, and 1 mg/L haemin.
4. The method of clause 2, wherein SHI medium comprises about 5.0 g/L tryptone, about 10.0 g/L peptone, about 2.5 g/L type III porcine mucin, about 5.0 g/L yeast extract, about 1.0 mg/L Vitamin K (Sigma-Aldrich), about 2.5 g/L KCl, about 5.0 mg/L haemin, about 0.174 g/L L-arginine, about 0.06 g/L urea, about 5% v/v sheep blood, and about 10.0 mg/L N-acetylmuramic acid.
5. The method of clause 1, further comprising step a-1) prior to step a):
a-1) providing a flow cell and at least one hydroxyapatite (HA) disc.
6. The method of clause 5, further comprising step a-2) after step a-1):
a-2) sterilizing the flow cell.
7. The method of clause 6, further comprising step a-3) after step a-2):
a-3) filling the flow cell with the growth medium devoid of sucrose for about 24 hours to allow pellicle formation.
8. The method of clause 1, further comprising step a-4) prior to step a):
a-4) mixing the dental plaque with a phosphate buffer solution (PBS) to form a first mixture.
9. The method of clause 8, further comprising step a-5) after step a-4):
a-5) adding the mixture to the growth medium to form a second mixture.
10. The method of clause 9, further comprising step a-6) after step a-5):
a-6) inoculating the second mixture into the flow cell.
11. The method of clause 10, further comprising step a-7) after step a-6):
a-7) flowing the flow cell with the growth medium.
12. The method of clause 11, further comprising step a-8) after step a-7):
a-8) growing the biofilm on the HA disc for at least 10 days at about 36° C.
13. The method of clause 1, wherein at least 60% of the cells in the biofilm are viable, or wherein at least 70% of the cells in the biofilm are viable.
14. The method of clause 1, wherein the biofilm comprises at least 200 bacterial species, or wherein the biofilm comprises at least 220 bacterial species, or wherein the biofilm comprises at least 240 bacterial species, or wherein the biofilm comprises about 252 bacterial species.
15. The method of clause 14, wherein the growth medium is ASM.
16. The method of clause 1, wherein the biofilm comprises aerobic and/or anaerobic bacteria.
17. The method of clause 16, wherein the anaerobic bacteria comprises Tannerella forsythia, Porphyromonas gingivalis, or a combination thereof.
18. The method of clause 1, wherein the collected dental plaque in step a) comprises about 312 bacterial species.
19. The method of clause 1, wherein the biofilm reduces the diversity of the bacterial species by at least 10% relative to the collected dental plaque.
20. The method of clause 1, wherein the biofilm reduces the diversity of the bacterial species by about 10% to about 40% relative to the collected dental plaque, or wherein the biofilm reduces the diversity of the bacterial species by about 10% to about 22% relative to the collected dental plaque.
21. The method of clause 1, wherein the microbiome of the biofilm comprises at least one bacterial species that does not exist in saliva or in the dental plaque.
22. The method of clause 1, wherein the microbiome of the biofilm has reduced abundance in Actinobacteria, Fusobacteria, or a combination thereof relative to the microbiome of the collected dental plaque.
23. The method of clause 22, wherein the growth medium is ASM.
24. The method of clause 1, wherein the microbiome of the biofilm has increased abundance in Bacteriodetes, Firmicutes, and a combination thereof relative to the microbiome of the collected dental plaque.
25. The method of clause 23, wherein the growth medium is ASM.
26. The method of clause 1, wherein the microbiome of the biofilm has increased abundance in Bacilli spp., Haemophilus parainfluenzae, or a combination thereof relative to the microbiome of the collected dental plaque.
27. The method of clause 26, wherein the growth medium is ASM.
28. The method of clause 1, wherein the microbiome of the biofilm has reduced abundance or removed the abundance of oral pathogens.
29. The method of clause 26, wherein the oral pathogen requires human tissue to grow.
30. The method of clause 27, wherein the oral pathogen comprises Porphyromonas gingivalis, Treponema denticola, or Tannerella forsythia, or any of the combinations thereof.
31. The method of clause 1, the biofilm reduces the proliferation of acidogenic and aciduric species or the proliferation of obligate anaerobic species.
32. The method of clause 1, the biofilm reduces the proliferation of Streptococcus mutans.
33. The method of clause 1, wherein the thickness of the biofilm is in the range of about 25 μm to about 55 μm.
34. The method of clause 1, wherein the biovolume of the biofilm is in the range of about 4 μm³ to about 8 μm³
35. The method of clause 1, wherein the donor is a human.
36. The method of clause 33, wherein the donor is a healthy human.
37. The method of clause 1, wherein the recipient is a human.
38. The method of clause 35, wherein the receipt is a patient.
39. A method for modulating the oral microbial composition of a recipient, comprising:
a) collecting dental plaque from a donor;
b) culturing a biofilm in vitro in a medium to modulate the oral microbial composition of the collected dental plaque from step a); and
c) transplanting the biofilm into the oral cavity of a recipient.
40. The method of clause 37, comprising any one of embodiments 2-37.
41. A method for treating cancer, mucositis, halitosis, or dry mouth according to any one of embodiments 1-37.
42. A modulated oral microbial composition prepared by:
a) collecting dental plaque from a donor; and
b) growing a biofilm in vitro in a growth medium to modulate the microbial composition of the collected dental plaque from step a).
43. The modulated microbial composition, comprising any one of embodiments 2-37.
44. A system comprising a flow cell and at least one hydroxyapatite (HA) disc for use to modulate an oral microbial composition.
45. The system of clause 44, wherein the flow cell is 3D printed.
46. The system of clause 44, wherein the flow cell mimics the salivary flow and environment of the human mouth.
47. The system of clause 46, wherein the flow cell is configured to move the growth medium at rates and in dispersion patterns that mimic how saliva spreads in the mouth around teeth.
48. The system of clause 47, wherein the HA disc is placed at the bottom of the flow cell.
49. The system of clause 47, wherein the HA disc is configured to allow a dental or oral biofilm to grow.

Fecal Microbiome Transplantation (FMT) in the gastrointestinal tract has been shown to be 81% effective in treating specific infectious diseases, such as Clostridium difficile infection, and up to 60% effective when paired with dietary interventions in diminishing clinical symptoms of inflammatory diseases, such as inflammatory bowel disease. However, similar technology has not yet been developed in the mouth, largely due to current dogmas in the oral microbiology field—dogmas that our approach contradicts. For example, the dental research community reported that the oral microbiome did not respond to changes in diet, as cultured oral microorganisms (mainly Streptococcus species) fed from salivary proteins produced and not on fleeting dietary nutrients. This suggests that oral microbes would not change or be different in people across different states of health. Further, oral microbiologists had shown that there was only one type of oral microbial community present, which was largely based on culturing-dependent research done exclusively in white, western European populations. The work in ancient and modern populations and research from other groups has shown that these dogmas are simply not true. In fact, the present application identified 125 oral species in adult Aboriginal Australian oral microbiomes that had not yet been described in the Human Oral Microbiome Database (HOMD; the current ‘gold standard’ for identifying oral microorganisms)—highlighting the magnitude of this inaccuracy. The present application leverages the differences between human populations to ensure the growth and transplanting of unique microbial communities.

The present application has modulated a microbial composition collected from healthy donors. The original oral microbial composition was obtained from the dental plaque of healthy donors and then modulated by growing a biofilm in vitro in a growth medium. The modulated oral microbial composition has a different microbial diversity than the original oral microbial composition. In the modulated oral microbial compositions, the abundance of some bacterial species changed, some new bacterial species were present, and some original bacterial species were no longer present. Then, the modulated oral microbial composition was transplanted to the recipient's oral cavity to treat dental caries or a periodontal disease.

The previous most robust model is disclosed in Edlund, A. et al. An in vitro biofilm model system maintains a highly reproducible species and metabolic diversity approaching that of the human oral microbiome. Microbiome 1, 25 (2013). The model utilizes saliva to inoculate or seed biofilms, which contain fewer anaerobic species. In contrast and advantageously, by collecting, homogenizing, and propagating dental plaque, the present application is able to obtain aerobic and anaerobic species, species routinely found in dental plaque, as well as other species and a modulated composition that are only found in the dental biofilm (and not saliva).

### EXAMPLES

The following examples are provided to further describe some of the embodiments disclosed herein. The examples are intended to illustrate, not to limit, the disclosed embodiments.

### Example 1. 3D Printed In Vitro Flow Cells

The oral microbiota consists of several distinct microbial communities in the mouth (i.e., those associated with teeth, gingival sulcus, attached gingiva, tongue, cheek, lip, and hard and soft palate). Although >700 oral bacterial species have been described in different human populations, each person maintains ˜200 bacterial species. Many oral microbes are shared within a population, as they are vertically inherited or transferred from a primary caregiver (e.g., mother) early in life. There is also evidence that the environment, smoking, salivary gland function, oral hygiene, and diet also play secondary roles in shaping oral microbiota. Dental biofilms present on hard tissues and restorative biomaterials make up a large proportion of oral microbiota; the most prolific is dental plaque that accumulates on tooth enamel. Plaque biofilms develop through interactions between microorganisms, host factors (e.g., salivary flow rate and buffering capacity), and dietary inputs (e.g., fermentable carbohydrates), making them difficult to fully reproduce in vitro. Plaque biofilms are also robust and notoriously difficult to modulate or ‘treat’ because the microbes are enmeshed in a protective extracellular matrix, making them recalcitrant to antibiotics.

To better understand oral biofilm physiology, several different types of biofilm growth chambers exist. Biofilm growth systems are generally divided into two major groups: open or closed, depending on their nutritional availability. In a closed batch culture system, nutrients are consumed over the duration of the experiment. The Zurich biofilm model is a key example of a closed biofilm model and only uses six bacterial species: Actinomyces naeslundii, Candida albicans, Fusobacterium nucleatum, Streptococcus oralis, Streptococcus sobrinus, and Veillonella dispar. Considering that supragingival plaque microbiota can contain >200 species, the present application provides a model that offers insights into complex oral microbial ecosystems. Open culture biofilm models are more likely to reflect conditions in the mouth, as nutrients are supplied continuously, similar to the human body. However, open systems also typically only employ less than 20 bacterial species and therefore do not accurately reflect oral microbial diversity in vivo.

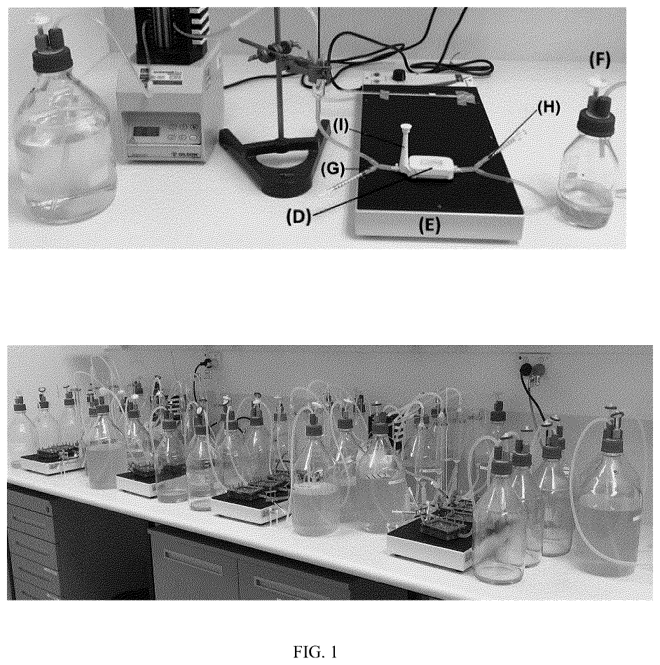

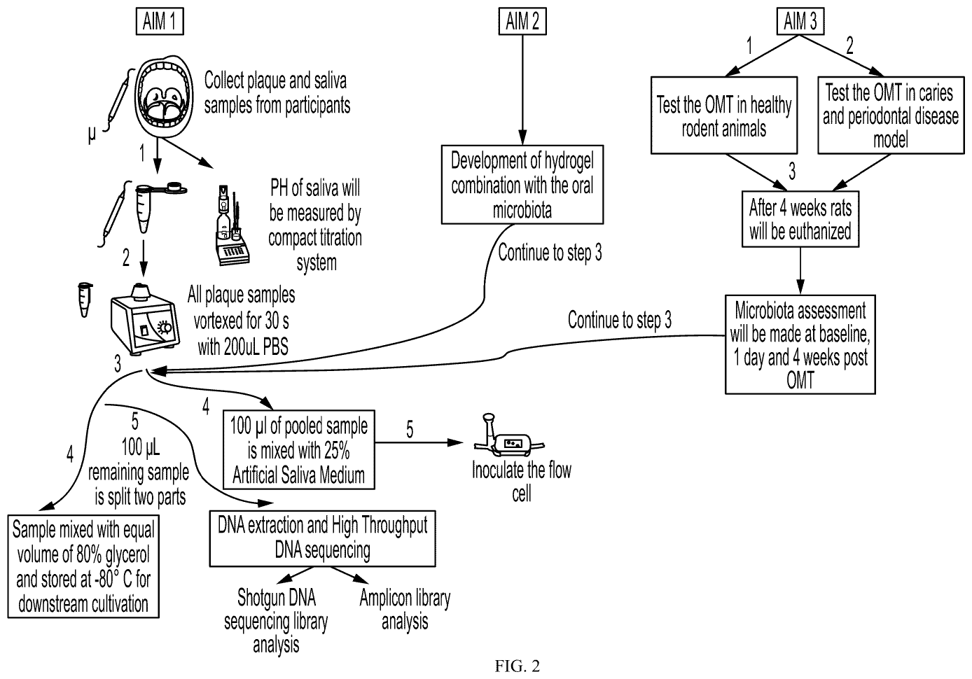

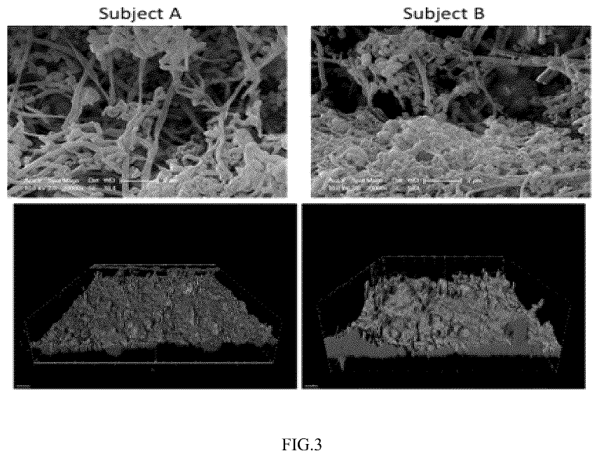

The present application provides an in vitro model that was developed to assess and grow plaque biofilms on hydroxyapatite (HA) disks (FIG. 1). The model combines state-of-the-art 3D printing, high-throughput DNA sequencing, and confocal microscopy. The model grows dental plaque directly from a donor, and therefore more closely reflects ‘real-life’ diversity of in vivo oral biofilms (>250 species), allowing for the manipulation of the environmental conditions, including pH, nutrient availability, and microbial composition to assess how biofilms change and adapt in real-world conditions. The system is also well suited for high-throughput DNA sequencing and other analyses (e.g., metaproteomics or metabolomics) and can be increased in size and parallelized (FIG. 2) to maximize experimental timing and growth.

### Example 2. Dental Plaque Collection from 100 Ultra-Healthy Donors

Dental plaque samples employed in this project were collected as part of a National Health and Medical Research Council grant in Australia to develop OMT therapy (APP1187737). Sample collection was granted by the University of Adelaide Human Research Ethics Committee in 2017 (H-2017-108). Dental plaque was collected from 100 ultra-healthy donors who had no reported chronic inflammatory conditions, gingivitis, caries, periodontal disease, or diabetes, as well as not be currently pregnant or prescribed antibiotics. A qualified dentist performed an oral health assessment and then collected supragingival plaque samples with a sterile Gracey curette from the buccal surface of two incisors and the mesio-buccal surface of the maxillary first and second molars.

### Example 3. Growth of Donor Material In Vitro

All plaque from that donor was vortexed together in a single, sterilized 1.5 mL tube containing 200 μl PBS (the ‘pooled’ sample). 100 μl of the pooled sample was mixed with 1000 μl of 25% Artificial Saliva Medium (ASM) and used to inoculate the flow cell (FIGS. 1 and 2). Fourteen days after inoculation, hydroxyapatite discs coated in donor biofilm were removed from the flow cell and were assessed using microscopy and DNA sequencing and were preserved for downstream use using two methods: 1.) mixed within 40% glycerol and phosphate saline buffer (PBS) and frozen at −80° C. or lyophilized and stored at −80° C.

The biofilms from these ultra-healthy donors were assessed after they were grown in the in vitro system for 14 days. The bacteria reassembled into biofilms in the in vitro system were evenly distributed across the surface of the HA discs. At 20,0000× magnification using Scanning Electron Microscopy (SEM), the well-established biofilm predominantly consisted of cocci-shaped bacteria. The living cells were characterized using Confocal Laser Scanning Microscopy (CLSM), revealing that the established biofilm consisted of an average of 73% live and 27% dead bacteria, with an average of >1×104 living cells retained on each of the 16 discs after 14 days. Overall, live oral biofilms can be reassembled in our in vitro system.

The biofilms were assessed from these ultra-healthy donors in Example 1 after they were grown in the in vitro system for 14 days. The in vitro flow cell can support living biofilms for at least 14 days. The bacteria reassembled into biofilms in the in vitro system were evenly distributed across the surface of the HA discs. At 20,0000× magnification using Scanning Electron Microscopy (SEM), the well-established biofilm predominantly consisted of cocci-shaped bacteria. The living cells were characterized using Confocal Laser Scanning Microscopy (CLSM), revealing that the established biofilm consisted of an average of 73% live and 27% dead bacteria, with an average of >1×104 living cells retained on each of the 16 discs after 14 days. Overall, live oral biofilms can be reassembled in the in vitro system.

The microbial composition of these in vitro biofilms was examined after 14 days of growth and compared to the initially donated plaque (inoculate). The composition of the inoculate and the mature biofilm significantly differed (Bray Curtis; PERMANOVA test; pseudo-F=8.31; p=<0.001). An average of 252 species were detected in each plaque sample after 14 days of growth, suggesting that this methodology is an effective way to assess and grow highly diverse in vitro biofilms for OMT transplantation. The diversity of the plaque biofilm after 14 days of growth was also significantly lower (Shannon's Diversity; Kruskal Wallis; H=10.19; p<0.001). This change was linked to a significant decrease in five Actinobacteria species and the entire loss of both Synergistetes and Spirochetes phyla. As some Spirochetes (e.g., Treponema denticola) are linked to poor oral health outcomes, the disappearance of these species in the in vitro system is a positive outcome.

### Example 4. OMT Application of Modulated Microbial Composition Grown on a Biofilm Reduced Caries Formation in a Rat Caries Model

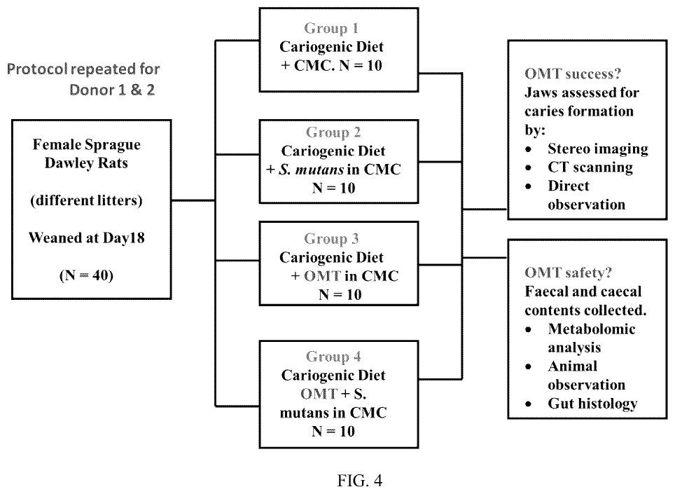

The six-week-old Sprague-Dawley rats were co-housed to assimilate their microbiota. The protocol is described in FIG. 4. The rats were additionally inoculated with Streptococcus mutans, which has been shown to cause caries in this rat model. The sample of fresh dental plaque from healthy donor rats was applied to grow biofilms as described in Example 3 for 10 days. The OMT was performed to transplant the biofilm to the recipient rats as described in FIG. 4.

Microbiota assessments were made at baseline, 1 day after the final OMT treatment, and 4-weeks post-OMT, using microscopy and DNA sequencing. A behavioral and physical health check of the animals was done weekly and at the end of the study. The animals were replaced if there were any deviations from normal healthy behavior. To collect microbiota during the experiment, the oral cavity of an anesthetized mouth was swabbed for 30 sec using nylon swabs, over the tongue, buccal areas, gingiva, palate, and tooth surfaces. Swabs were placed into a sterile PBS buffer, alongside a negative control, and analyzed immediately or frozen at −80 C until DNA extraction. At the end, rodents were swabbed a final time, and concluding oral samples were taken for microscopy and DNA analysis.

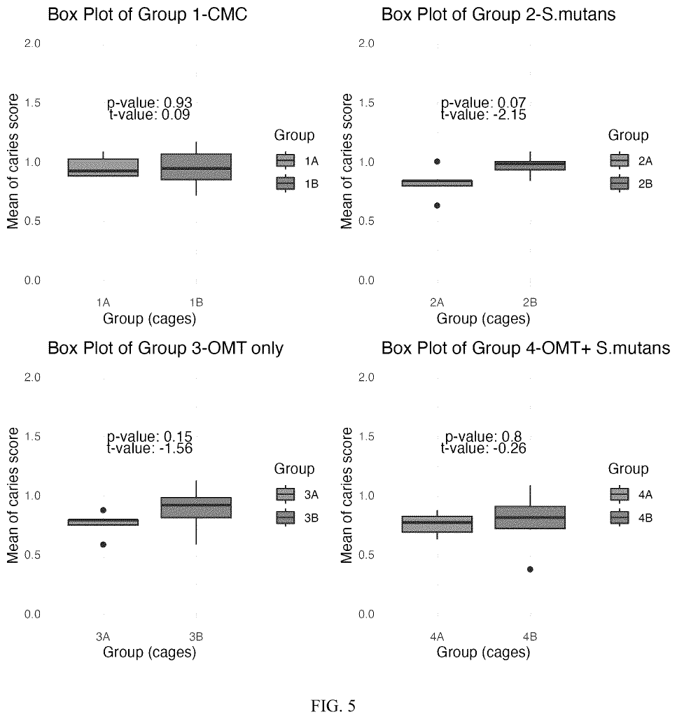

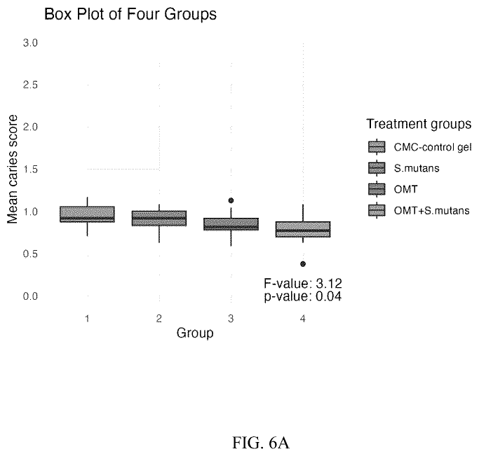

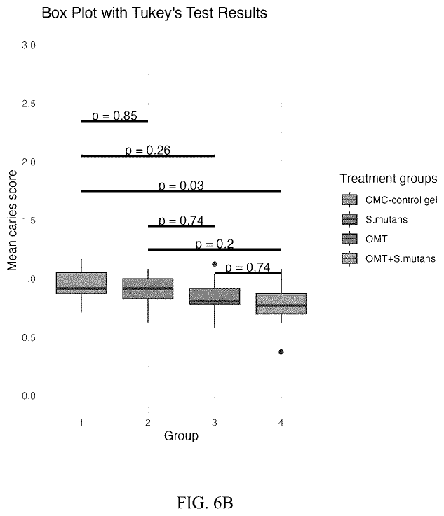

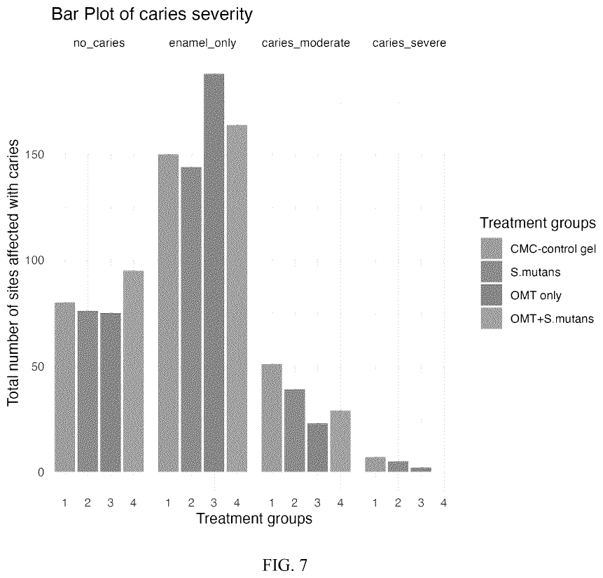

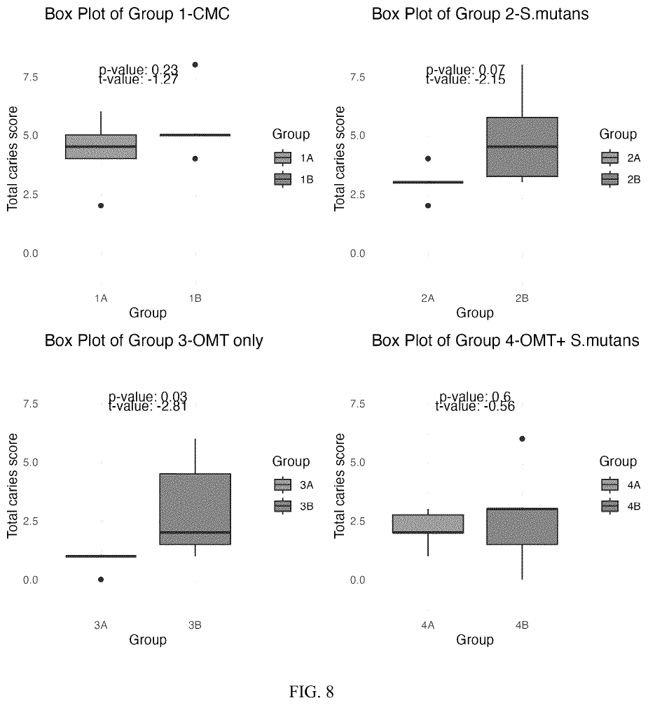

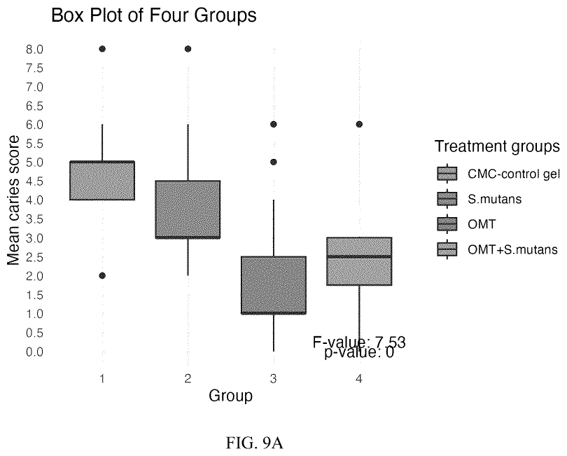

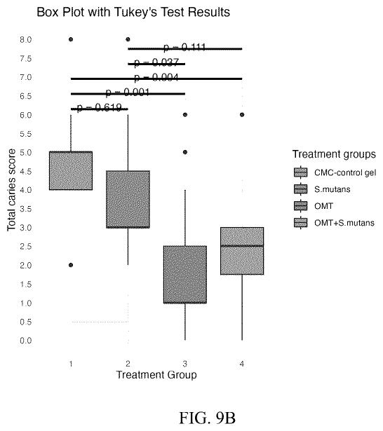

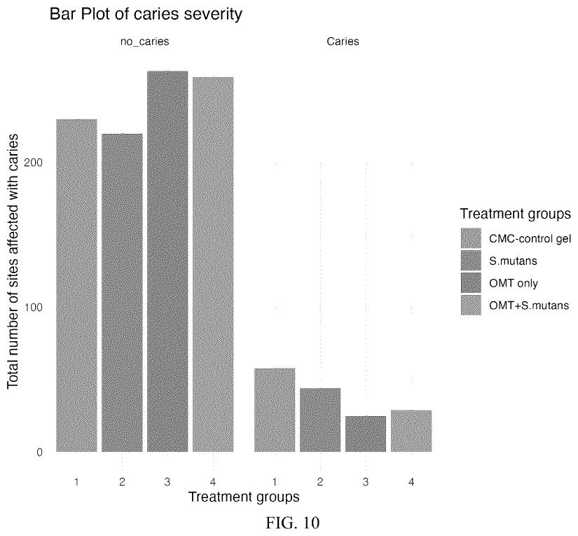

Dental plaque from two donors was used and the OMT was performed in a rat caries model. An OMT was performed by brushing donor plaque suspended in a hydrogel onto the rat's teeth for three consecutive days. The formation of caries was then examined in rats at the completion of the experiment. The caries analyzed here were assessed by blinded dental clinicians using both the defleshed rat skeletal elements and micro-CT scanning to ensure that the scoring was done objectively. FIGS. 5-7 use a caries scoring system on a scale of 1-5. FIGS. 8-10 use a binary caries scoring system (either having caries (1) or no caries (0)). Both groups of rats receiving OMTs were able to reduce caries formation significantly from the control groups (p=0.001 and p=0.004 for OMT and OMT with S. mutans groups using a Tukey's test). The OMT from two different donors was able to significantly reduce caries formation in a rat model, suggesting that OMT is effective at partially reducing caries formation or preventing carious lesions in some individuals.

### Example 5. An In Vitro Biofilm Model of the Human Supra-Gingival Microbiome

### Collection of Supra-Gingival Plaque

Dental plaque was collected from six healthy volunteers (aged 25-60 years). Prior to plaque collection, the tip of a sterile Gracey curette (5/6) (Henry Schein, NSW, Australia) was washed in 200 μL sterile PBS (pH 7.4) and stored at −80° C. to monitor contaminant DNA. Plaque was collected from the buccal interproximal sites of the first two incisors (#31, #32) and the mesio-buccal surface of maxillary first and second molars (#36, #37). Plaque samples from each volunteer were pooled into 200 μL sterile PBS.

### Flow Cell and Inoculation

FCs were 3D printed in polypropylene to enable sterilization. The flow rate of the medium was adjusted to 2/5 the total volume of the FC. Each FC contained five hydroxyapatite (HA) discs (D=5 mm×H=2 mm; Clarkson Chromatography Products, PA, USA).

Following sterilization, FCs were filled with either 25% ASM or SHI medium devoid of sucrose 24 hours prior to inoculation to allow pellicle formation and confirm sterility. Briefly, ASM contained 0.50 g/L tryptone (Oxoid, England), 0.50 g/L neutralized bacteriological peptone (Oxoid), 0.625 g/L type III porcine gastric mucin (Sigma-Aldrich, Germany), 0.25 g/L yeast extract (Oxoid), 0.05 g/L KCl, 0.05 g/L CaCl₂), 0.088 g/L NaCl, and 1 mg/L haemin (Sigma-Aldrich). SHI contained 5.0 g/L tryptone, 10.0 g/L peptone, 2.5 g/L type III porcine mucin, 5.0 g/L yeast extract, 1.0 mg/L Vitamin K (Sigma-Aldrich), 2.5 g/L KCl, 5.0 mg/L haemin, 0.174 g/L L-arginine (Sigma-Aldrich), 0.06 g/L urea (Sigma-Aldrich), and supplemented with 5% v/v sheep blood (Oxoid) and 10.0 mg/L N-acetylmuramic Acid (Sigma-Aldrich). Three of the six donors for growth in ASM (1-6) also donated plaque for growth in SHI (4-6). The fresh plaque was vortexed in PBS for 30 s, and 100 μL was added to 1 mL of either ASM or SHI and used to inoculate the FC. Medium flow began after 24 hours, and the biofilm was grown at 36° C. for 14 days. 100 μL of the planktonic culture was then removed for sequencing, and each HA disc was removed and placed into a separate sterile 1.5 mL tube. Two discs were used for scanning electron microscopy (SEM) and confocal laser scanning microscopy (CLSM), while three discs were kept for High Throughput Sequencing (HTS) (Table 1).

TABLE 1
List of samples collected during the study. Biological
samples, 1-5; Control samples, 6-9.

| Sample | | |
|---|---|---|
| number | Sample name | Description |
| 1. | Collected plaque | Plaque collected from the donor and |
| | | placed into PBS |
| 2. | Inoculated plaque/T0 | Plaque sample mixed with nutrient |
| | | medium prior to FC inoculation |
| 3. | T14 biofilms | Biofilms formed on HA discs after |
| | | 14 days |
| 4. | Planktonic cells | Planktonic cells taken from the FC |
| | | after 14 days |
| 5. | Spent medium | Medium outflow (waste) from the FC |
| | | collected for 14 days |
| 6. | FC control | Medium collected from FC prior to |
| | | plaque inoculation |
| 7. | Medium control at T0 | Nutrient medium collected at T0 |
| 8. | Medium control at T14 | Nutrient medium collected at T14 |
| 9. | PBS control | PBS collected prior to the start |
| | | of the experiment |

### Biofilm Imaging

### SEM

Each disc was prepared for SEM, and coated with 2 nm platinum, and imaged with a SEM-FEI Quanta 450 FEG. Due to variations in the density of biofilms, micrographs were imaged at three random positions on discs at 1,000×, 5,000× and 20,000× magnification.

### CLSM

Each disc was washed in sterile PBS three times for 10 minutes to remove unbound cells. Biofilms were stained using a LIVE/DEAD™ BacLight™ kit (Invitrogen, Vic, Australia), following the manufacturer's instructions. Images were captured at three random positions using an Olympus FV3000 microscope at 60× magnification using the dimensions of 1024 pixels for both axes. Each stack was acquired using a z-step size of 0.5 μm. Images were processed using IMARIS software (Bitplane, Zurich, Switzerland, version 7.6) for viability, average biofilm volume (μm³), and thickness (μm). Collated cells were excluded from the analysis.

16S rRNA Marker Gene Sequencing

DNA was extracted from 100 μL taken from controls; collected and inoculated plaque; three HA discs; and planktonic cells (Table 1) using the Qiagen DNeasy PowerSoil® Kit (Qiagen, Maryland, USA), following the manufacturer's instructions. Extraction blank controls (EBCs) were added at the start and end of each extraction to monitor laboratory contamination.

DNA sequencing libraries were generated by amplifying the V4 hypervariable region of 16S rRNA gene, as previously described, using an Invitrogen Platinum High Fidelity DNA polymerase. Thermocycling conditions consisted of 6 minutes of denaturation at 95° C.; 38 cycles of 95° C. for 30 s, 50° C. for 30 s and 72° C. for 90 s; and a final extension of 60° C. for 10 minutes; one no-template control (NTC) was added per amplification round. Samples were quantified using the Invitrogen Qubit dsDNA BR assay (Life Technologies), cleaned using 1.1× Axygen AxyPrep Mag™ PCR Clean-up beads, quantified using the D1000 reagents on the Tapestation (Agilent, Santa Clara, CA, USA), and equimolar pooled at 5 nM for sequencing at the South Australia Health and Medical Research Institute (SAHMRI, Adelaide, Australia) on an Illumina MiSeq using 150 bp paired-end sequencing. The demultiplexed sequencing data was imported into the Quantitative Insights Into Microbial Ecology (QIIME2-2021.4). Deblurring was performed on merged, demultiplexed, paired-end sequence reads that were quality filtered, and DNA sequences were denoised in amplicon sequence variants (ASVs) and trimmed to 120 bp.

Contaminants from laboratory sources (EBCs and NTCs) and FC controls were removed using the R package decontam implemented in phyloseq R using the prevalence-based method. Decontam score thresholds were chosen based on a histogram of decontam scores for each filtration step.

### Visualization and Statistical Analysis

Diversity and compositional analyses were done using QIIME2. Sequences from each sample were rarefied to 1,462 and 26,608 sequences, as the highest number of sequences from any SHI or ASM sample, respectively. α-diversity was calculated using the observed features metric of each sample. The β-diversity of the samples was analyzed using weighted and unweighted UniFrac (phylogenetic matrices), Jaccard, and the Bray Curtis indexes and visualized using principal coordinates analysis (PCoA) plots with EMPeror. A Kruskal-Wallis test was used to assess significant differences in α-diversity between sample types. Significant differences in β-diversity were assessed using PERMANOVA. A corrected p-value of <0.05 was considered significant. Linear discriminant analysis (LDA) effect size (LEfSe) was used to detect significantly abundant bacterial species between groups; an LDA score of >2 was used as the significance threshold. Significantly abundant taxa were visualized in a heat map using the ‘heatmap’ function in QIIME2.

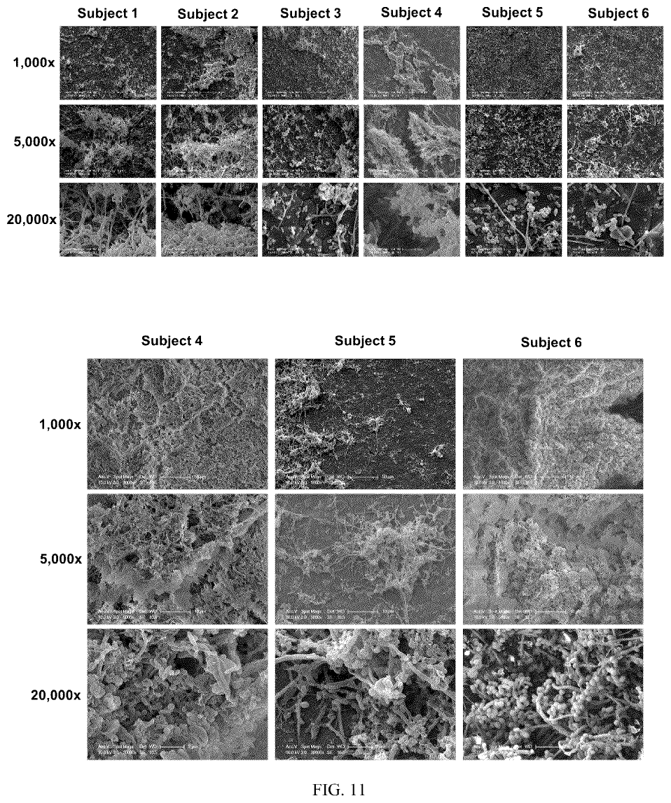

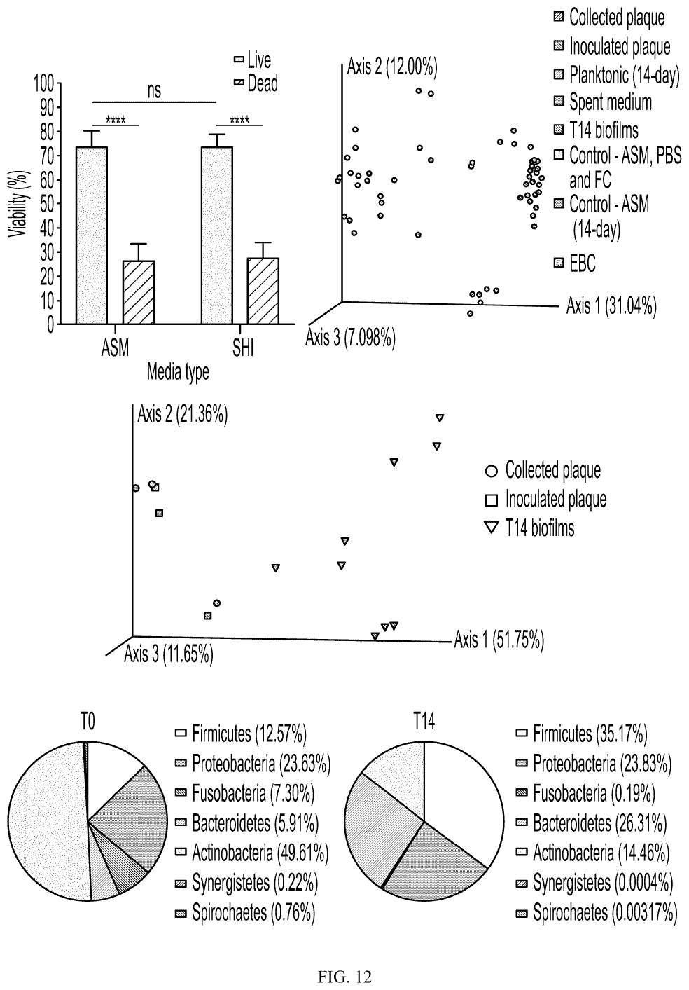

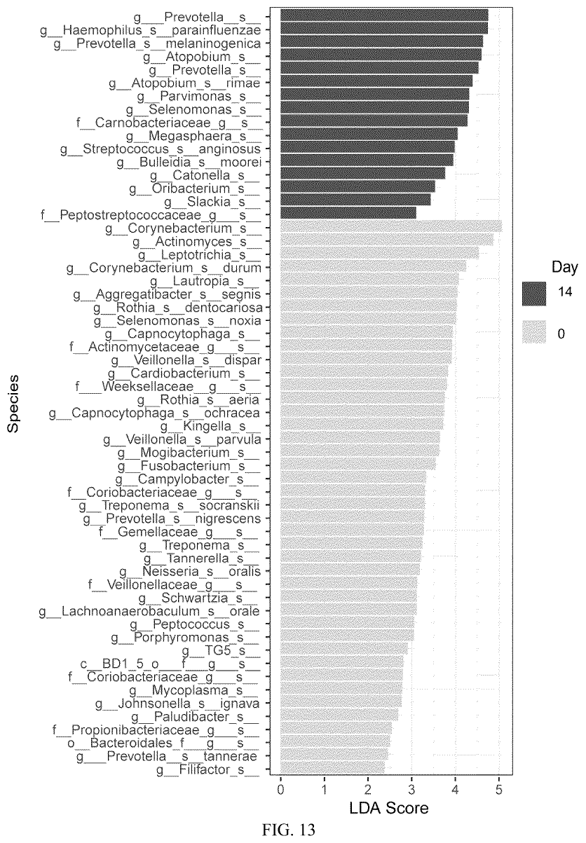

There is no significant difference in viability, thickness and volume of biofilms grown in ASM and SHI. Qualitative SEM assessment of biofilms grown on HA discs in either ASM or SHI were predominantly of rod- and cocci-shaped bacteria that grew in ‘islands’ and varied in biofilm density between donors (FIG. 11A, B). CLSM analysis revealed an average 1.57×10⁴±6.08×10³ and 3.48×10⁴±1.74×10⁴ live and dead cells from plaque grown in ASM and SHI, and importantly, >70% of cells were viable for biofilms grown in ASM and SHI (FIG. 12A). The number of viable cells was not significantly different for biofilms grown in ASM (1.15×10⁴±3.91×10³) compared to SHI (2.45×10⁴ 1.04×10⁴). Lastly, the average thickness and biovolume of ASM-grown biofilms were 30.88±6.11 μm and 4.36×10⁵±8.48×10⁴ μm³, respectively, while the SHI-grown biofilms were 45.56±9.01 μm and 6.36×10⁵±1.25×10⁵ μm³, respectively. Despite inter-donor variability, no significant difference was observed in biofilm thickness and volume between SHI or ASM across all donors.

There are significant differences in microbial composition between biological and control samples in ASM and SHI. The initial analysis aimed to reduce the impact of contaminant DNA introduced during DNA extraction and library preparation, as well as the laboratory environment during collection and growth in the FC system. The compositions of the controls alongside the biological samples were examined by conducting PCoA of unweighted UniFrac values (FIG. 12B). The microbial composition of laboratory controls (EBCs/NTCs) was significantly different from biological samples, including spent medium, planktonic cells, inoculated samples, and biofilms grown over 14-days in ASM (PERMANOVA, pseudo-F; test: 21.51; p<0.001). Contaminants identified in the EBCs/NCTs accounted for 30.55% (42 species) of the total species and were removed from the data using the decontam package. The composition of FC controls (Table 1) was also significantly different from biological samples (PERMANOVA, pseudo-F; test: 17.99; p<0.001). The contaminant ASVs present in the FC controls were removed using decontam, which accounted for 17.63% (35 species) of the remaining sequences in all biological samples post filtering with EBCs.

Similar to ASM, the unweighted UniFrac calculated compositions of the laboratory and FC controls were significantly different from biological samples grown in SHI (FIG. 15A) (PERMANOVA, pseudo-F; test: 4.18; p<0.001 and pseudo-F; test: 5.27; p<0.001, respectively). Contaminants identified in the EBCs/NCTs accounted for 17.63% (42 species) of the total species and were removed from the data using the decontam package, while contaminant ASVs present in the FC controls accounted for an additional 17.63% (65 species), respectively post filtering with EBCs. α-diversity was reduced in T14 biofilms grown in ASM

The α-diversity was compared between collected plaque, inoculated plaque (T0), and T14 biofilms (Table 1). Collected and inoculated plaque samples had an average of 312 species per donor, and after 14 days of growth in vitro, an average of 252 species were recovered from each donor biofilm (overall 291 overall species recovered). This was a significant decrease in overall α-diversity compared to the original inoculum (Kruskal-Wallis Pairwise; H test: 6.25, p<0.05), but the donor specific α-diversity signatures are retained in all sample groups (Kruskal-Wallis; All Groups; H test: 13.05, p>0.05). Further, 17 species were lost between collection and inoculation, but the overall α-diversity was not significantly different between collection and inoculation (Kruskal-Wallis Pairwise; H test: 1.19, p>0.05).

### β-Diversity was Maintained Between Collected and Inoculated Plaque to Mature Biofilms in ASM

The compositional shifts were examined from plaque collection through to T14 biofilms grown in ASM. Collected and inoculated plaque samples clustered closely together, compared to T14 biofilms. There was no significant difference in β-diversity between the collected and inoculated samples (PERMANOVA test; pseudo-F; test: 0.23; p>0.05). However, significant differences in bacterial composition were observed after 14 days in vitro. Inoculated samples and T14 biofilms were significantly different across all six donors (PERMANOVA test; pseudo-F; test: 5.68; p≤0.001). Nevertheless, the microbial community composition of biofilms grown on three discs from the same FC clustered closely according to each donor.

### α- and β-Diversity Shifts in Biofilms Grown in SHI

Similar to ASM, the α-diversity was compared between inoculated plaque, and T14 biofilms grown in SHI. Inoculated plaque and T14 biofilms had an average of 103 and 61 species, respectively per donor, which was markedly fewer than donated plaque that was used to inoculate ASM-filled FCs. Overall, a total of 124 and 95 species were identified from inoculated plaque and T14 biofilms. This increase in overall α-diversity compared to the inoculated plaque, was significantly different (Kruskal-Wallis All Groups; H test: 7.44, p<0.05). Similar to ASM, no compositional shifts were observed in inoculated plaque compared to T14 biofilms grown in SHI (FIG. 15C; PERMANOVA test; pseudo-F; test: 1.36; p>0.05).

### Reduction in Significantly Abundant Species at T14 Biofilms Compared to Collected and Inoculated Plaque in ASM

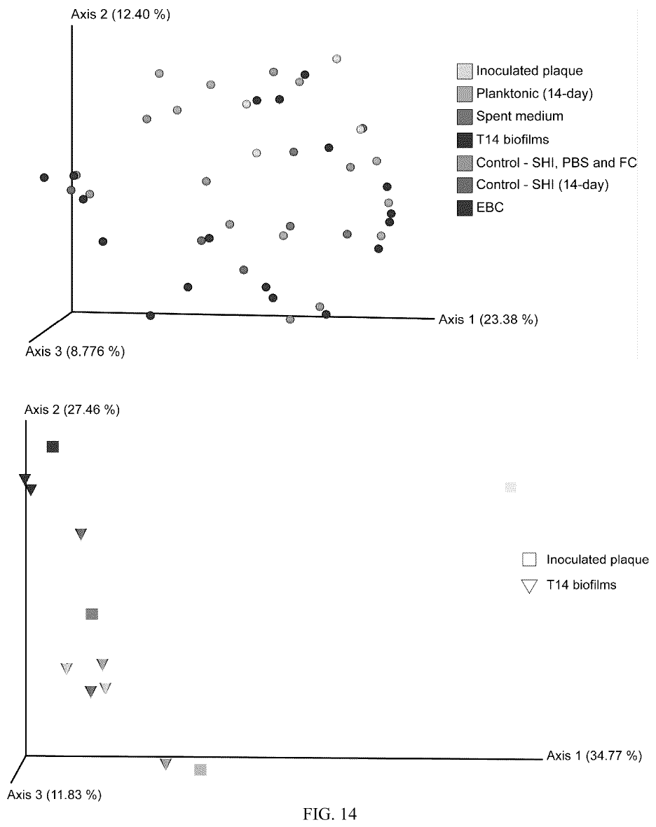

Taxonomic data plotted at a phylum level revealed few differences in relative abundances between collected and inoculated samples (FIG. 12D). However, when inoculated samples were compared to T14 biofilms, the relative abundance of Firmicutes and Bacteriodetes increased while Actinobacteria and Fusobaceteria decreased. Specifically, LefSe identified 42 significantly abundant ASVs that were higher in abundance at the time of inoculation (T0) compared to T14, belonging to Firmicutes (n=11), Actinobacteria (n=9), Proteobacteria (n=6), Fusobacteria (n=2), Bacteriodetes (n=1), Tenericutes (n=1), GN02 (n=1), Spirochaetes (n=1) and Synergistetes (n=1) phyla (FIG. 14). In contrast, 16 differentially abundant genera were more prevalent in T14 biofilms, Firmicutes (n=9), Actinobacteria (n=3), Bacteriodetes (n=3) and Proteobacteria (n=1). The reduction of Fusobacteria, Spirochaetes, and Synergistetes at 14 days is a positive result as these phyla are linked to poor oral health outcomes.

The emergence of innovative biofilm models, HTS, and improved cultivation methods for oral microbiota have contributed to our understanding of the role and function of oral bacteria. However, ˜250-300 bacterial species are thought to be maintained as a core microbiome in the oral cavity, and a significant challenge remains in reproducing this diversity in vitro. Visual analysis of biofilms was consistent with other research which found local variations in the density of biofilms grown on HA discs. These findings highlight the need to select an assortment of random sampling regions for biofilm analysis.

HTS indicated the overall richness and diversity of T14 biofilms grown with ASM was significantly decreased compared to inoculated plaque. Additionally, there was a significant alteration of taxa with five phyla between inoculated plaque and T14 biofilms. A similar shift was identified in bacterial composition at the phyla level, with the exception being TM7 bacteria, which was present only in the collected plaque samples in relatively low proportions (<1%). While a significant decrease in alpha diversity was observed for biofilms grown in ASM, an average of 252 bacterial species were detected in each plaque sample (275-244 species) after 14 days. This is the highest microbial diversity using an in vitro FC model.

Published ASM and SHI mediums both included sucrose; however, sucrose was excluded in this study because it favors the growth of cariogenic bacteria. CLSM analysis showed that there was no significant difference in biofilm viability, biovolume and thickness for SHI medium compared with ASM; further, the viability of biofilms was not influenced by the growth medium. Significant differences in bacterial composition between T0 and T14 in ASM were also not seen with SHI. Lastly, Bacilli spp. were also more prevalent after 14 days in SHI-grown biofilms. Additionally, the abundance of Haemophiluspara influenzae in donor 2 and 4 almost doubled by T14. Bacilli are significantly associated with periodontal health and H. parainfluenzae is shown to have anti-cancer (oral) properties in vitro.

The present application is capable of growing oral biofilms with the highest oral microbial diversity reported (average of 252 species) using ASM. This study also demonstrated the ability of ASM to maintain a higher species diversity in comparison to SHI medium. The present application can be used as an antimicrobial testing platform for antimicrobials, oral care products, natural and synthetic compounds, and oral microbiome transplant therapy.

### Example 6. Select Specific OMT Donor Plaque Targeted for Caries Prevention (Prophetic)

The most suitable OMT donor dental plaque will have no or limited quantities of Streptococcus mutans and other known oral pathobionts (i.e., Porphyromonas gingivalis, Treponema denticola, or Tannerella forsythia) and be able to buffer acid in the mouth (i.e., as examined by a glucose challenge test).

OMT therapy should not pose new health risks, as it is aimed to develop OMT technology that maximizes patient benefits and minimizes risks. Specifically, the top 20 donors in each group will be tested for potential pathogens linked to caries (i.e., S. mutans); If individuals do possess S. mutans, as confirmed by culture and genetic analysis, the genome of these S. mutans strains will be assembled to examine the presence of acid production capability and antimicrobial resistance genes. The genomics analysis of these strains will be used to guide this part of the selection process. Using shotgun sequencing, the donors will be screened for known microbes linked to other oral disease, such as periodontal disease (P. gingivalis, T. denticola, and T. forsythia), using high throughput shotgun sequencing and strain-level identification of bacterial species (i.e., StrainPhlAn). The presence of oral viruses to transmit via human saliva will be determined known (i.e., Epstein Barr virus, herpes viruses, etc.) and other known oral, gastrointestinal, or sexually transmitted viruses and bacteria (i.e., HIV, syphilis, etc.) will be determined.

Once donors are deemed safe for transplant, it will be examined regarding how different donor lifestyles may impact their ability to prevent caries. Decades of research have focused on how the consumption of dietary sugars (e.g., refined sugars in the form of soda, candy, etc) influences caries and cavity development via the oral microbiota, both on individual species (e.g., Streptococcus mutans) and the composition of the whole oral microbiota. As such, it is critical to examine how different donor diets may influence the microbial variation in our top donors from each group. For example, the microbiota from a donor who never consumes sugar should ideally maintain an oral microbiota that still has good buffering capacity against low pH even when placed in a donor that consumes large amounts of sugar. To examine this in the context of caries risk, the in vitro system will be used to test how microbial composition of donors with different diets and lifestyles grow in vitro and how buffer acid production (i.e., as measured by pH from the flow cell's outflow valve) during a glucose challenge; other sugars (e.g., glucose, lactose, fructose, or sucrose) can also be assessed in this system. For example, donor oral microbiota will be cultured using the in vitro flow cell system with the addition of 5%, 10% or 15% glucose to reflect diets that have higher levels of dietary sugar. Additionally, glucose will also be “pulsed” into the flow cell (i.e., added and then not added) to simulate dietary behavior over 24 hours and to monitor pH changes. The sugar can be added for a prescribed time (e.g., for one day at day seven of a time course) to see if a healthy microbiome is rapidly shifted and can re-establish. High-throughput DNA sequencing (16S rRNA amplicon sequencing) and pH will guide the selection of the 12 most suitable donors from each group. If fewer than 12 are identified, that number of donors will be proceeded. While it is possible that no donors can be identified, the preliminary data suggested that only 12% maintained S. mutans strains, and none had a history of oral infections, heightening our chances for success.

### Example 7. Assess Efficacy of OMT Therapy to Prevent Dental Caries in a Rat Model of Disease (Prophetic)

OMT therapy in a rat model of dental caries will reduce the formation of dental caries in a rat caries model. This will be achieved by altering the composition of the oral microbial community and changing the microbial activity on the tooth surface.

Using the 12 most donors from each group selected from Aim 1 (n=36), each donor will be examined in a rat model of dental caries. It is predicted that many, but not all, donors will prevent caries formation. Groups of 32 mice (n=16 in the control group and n=16 in the experimental group with OMT donation) will be run concurrently, and while it is tempting to increase the study size, this experimentation is difficult with larger cohorts of mice, as the feeding regimen required for this experiment (i.e., 5 times per day) is time-consuming. After the experimentation is finished, caries assessment will be completed on the three surfaces of all molar teeth by at least two standardized assessors. If the lesions are difficult to assess in all ciesanimals as observed in some younger rats in the preliminary data, microCT scanning can be employed by the Penn State Center for Quantitative Imaging to better assess the early stages of caries formation. To assess shifts in microbiota, the 16S rRNA will be used to screen all oral samples. The pre-OMT, day four (just post-OMT), and post-experimentation samples will be shotgun sequenced to verify strain maintenance throughout the procedure and also in cases where individual strain tracking is needed (i.e., in individuals with S. mutans or other pathobionts or where certain Streptococcus species cannot be distinguished between rat and donor using 16S rRNA). A portion of the unextracted shotgun sequenced samples will also be preserved and used to assess the metabolite profiles to examine functional shifts in the community.

### Example 8. Studies to Establish if OMT Therapy is Safe and has No Adverse Side Effects (Prophetic)

OMT therapy should not result in increased systemic inflammation (i.e., as observed by lumen histology or C-Reactive Protein production) or shifts in the gut microbiome diversity or function.

The additional samples collected during the rat caries model will be examined, including luminal samples, gut microbiome specimens, and blood. The rat lumen will be processed for histology to look for signs of inflammation. Gut microbiome samples (i.e., fecal pellets) collected before, immediately following the final OMT transplant, and at the conclusion of the study will be examined for microbial shifts (alpha and beta diversity), as well as metabolomic shifts. Significant shifts following OMT or at the conclusion of the study would be deemed as potential adverse effects and warrant further exploration. The rat blood obtained before, after OMT, and at the conclusion of the experiment for CRP levels will be examined; if elevated throughout the process, it will be deemed as a potential adverse effect. This work will set a precedent for downstream examinations looking at OMT therapy in other murine models of disease; for example, an OMT transplant should also not further exacerbate symptoms of other systemic diseases that can be associated with poor oral health (i.e., cardiovascular disease, diabetes, or rheumatoid arthritis, as assessed with specific murine models for those diseases).

### Example 9. Flow and Process for Rowing Unique Dental Plaque for Oral Microbiome Transplantation

1.) Set up 3D printed, sterilized flow cell for the growth of plaque and ensure the system is not contaminated by running for 8 days as a closed system. Ensure that hydroxyapatite (HA) discs are placed into the sterilized system, and select which medium should be used, such as artificial salivary medium (ASM) or others, such as SHI.
2.) Collect dental plaque from consenting donors.
3.) Homogenize dental plaque in 200 uL of sterile phosphate saline buffer by vortexing.
4.) Assess pH using a glucose challenge test.
5.) Mix 100 uL of homogenized dental plaque with 1 mL of 25 modified artificial salivary medium to create innoculate.
6.) Seed the sterilized flow cell system using 100 uL of inoculate through the input value.
7.) Mix the flow cell once each day for 10 days and ensure that liquid is flowing through the system.
8.) At the end of ten days of growth, open the flow cell, remove at least two HA discs, verify growth of live microorganisms using live:dead microscopy, and perform 16S ribosomal RNA sequencing to define the species that were maintained and grown in dental plaque.

### REFERENCES

1. Nath, S. et al. Development and characterization of an oral microbiome transplant among Australians for the treatment of dental caries and periodontal disease: A study protocol. PloS one 16, e0260433 (2021).
2. Drekonja, D. et al. Fecal Microbiota Transplantation for Clostridium difficile Infection. Ann Intern Med 162, 630-638 (2015).
3. Kedia, S. et al. Fecal microbiota transplantation with anti-inflammatory diet (FMT-AID) followed by anti-inflammatory diet alone is effective in inducing and maintaining remission over 1 year in mild to moderate ulcerative colitis: a randomized controlled trial. Gut 71, 2401-2413 (2022).
4. Morhart, R. E. & Fitzgerald, R. J. Nutritional determinants of the ecology of the oral flora. Dent Clin North Am 20, 473-489 (1976).
5. Wade, W. G. Resilience of the oral microbiome. Periodontol 2000 86, 113-122 (2021).
6. Stallard, R. E. A Textbook of preventive dentistry. (Saunders, 1982).
7. Campbell, K. Oral microbiome findings challenge dentistry dogma. Nature (2021) doi:10.1038/d41586-021-02920-w.
8. Handsley-Davis, M. et al. Heritage-specific oral microbiota in Indigenous Australian dental calculus. Evolution, Medicine, and Public Health 10, 352-362 (2022).
9. Edlund, A. et al. An in vitro biofilm model system maintaining a highly reproducible species and metabolic diversity approaching that of the human oral microbiome. Microbiome 1, 25 (2013).
10. Bagramian, R. A., Garcia-Godoy, F. & Volpe, A. R. The global increase in dental caries. A pending public health crisis. Am J Dent 22, 3-8 (2009).
11. Marsh and Martin's Oral Microbiology—6th Edition. https://www.elsevier.com/books/marsh-and-martins-oral-microbiology/marsh/978-0-7020-6106-6.
12. Borody, T. J. & Khoruts, A. Fecal microbiota transplantation and emerging applications. Nature Reviews Gastroenterology & Amp; Hepatology 9, 88 (2011).
13. Walls, A. W. G., Steele, J. G., Sheiham, A., Marcenes, W. & Moynihan, P. J. Oral Health and Nutrition in Older People. Journal of Public Health Dentistry 60, 304-307 (2000).
14. World Health Organization. Sugars and dental caries. Sugars and dental caries https://www.who.int/news-room/fact-sheets/detail/sugars-and-dental-caries (2017).
15. World Health Organization Oral health fact sheet. https://www.who.int/news-room/fact-sheets/detail/oral-health.
16. Righolt, A. J., Jevdjevic, M., Marcenes, W. & Listl, S. Global-, Regional-, and Country-Level Economic Impacts of Dental Diseases in 2015. J Dent Res 97, 501-507 (2018).
17. Tellez, M., Zini, A. & Estupinan-Day, S. Social Determinants and Oral Health: An Update. Curr Oral Health Rep 1, 148-152 (2014).
18. Sender, R., Fuchs, S. & Milo, R. Revised Estimates for the Number of Human and Bacteria Cells in the Body. PLoS Biol 14, (2016).
19. Turnbaugh, P. J. et al. The Human Microbiome Project. Nature 449, 804-810 (2007).
20. Turnbaugh, P. J. et al. An obesity-associated gut microbiome with increased capacity for energy harvest. Nature 444, nature05414 (2006).
21. Venter, J. C. et al. The Sequence of the Human Genome. Science 291, 1304-1351 (2001).
22. Kilian, M. et al. The oral microbiome—an update for oral healthcare professionals. British Dental Journal 221, 657-666 (2016).
23. Dewhirst, F. E. et al. The Human Oral Microbiome. J Bacteriol. 192, 5002-5017 (2010).
24. Pitts, N. B. et al. Dental caries. Nat Rev Dis Primers 3, 17030 (2017).
25. Selwitz, R. H., Ismail, A. I. & Pitts, N. B. Dental caries. The Lancet 369, 51-59 (2007).
26. Salli, K. M., Forssten, S. D., Lahtinen, S. J. & Ouwehand, A. C. Influence of sucrose and xylitol on an early Streptococcus mutans biofilm in a dental simulator. Arch. Oral Biol. 70, 39-46 (2016).
27. Oral health and dental care in Australia: key facts and figures 2015, Table of contents. Australian Institute of Health and Welfare https://www.aihw.gov.au/reports/dental-oral-health/oral-health-and-dental-care-in-australia-key-facts-and-figures-2015/contents/table-of-contents.
28. Nascimento, M. M. Oral microbiota transplant: a potential new therapy for oral diseases. J Calf Dent Assoc 45, 565-568 (2017).
29. Xiao, H. et al. Oral microbiota transplantation fights against head and neck radiotherapy-induced oral mucositis in mice. Comput Struct Biotechnol J 19, 5898-5910 (2021).
30. Tonetti, M. S., Jepsen, S., Jin, L. & Otomo-Corgel, J. Impact of the global burden of periodontal diseases on health, nutrition and wellbeing of mankind: A call for global action. J. Clin. Periodontol. 44, 456-462 (2017).
31. Zhao, Y. et al. Costs and benefits of community water fluoridation in remote Aboriginal communities of the Northern Territory. Australian Journal of Rural Health n/a.
32. Jamieson, L. M., Thomson, W. M. & McGee, R. An assessment of the validity and reliability of dental self-report items used in a National Child Nutrition Survey. Community Dentistry and Oral Epidemiology 32, 49-54 (2004).
33. Costello, E. K. et al. Bacterial Community Variation in Human Body Habitats Across Space and Time. Science 326, 1694-1697 (2009).
34. Shin, H. et al. Changes in the Eye Microbiota Associated with Contact Lens Wearing. mBio 7, e00198-16 (2016).
35. Cho, I. & Blaser, M. J. The Human Microbiome: at the interface of health and disease. Nat Rev Genet 13, 260-270 (2012).
36. Dewhirst, F. E. et al. The Human Oral Microbiome. J. Bacteriol. 192, 5002-5017 (2010).
37. Caufield, P. W., Ratanapridakul, K., Allen, D. N. & Cutter, G. R. Plasmid-containing strains of Streptococcus mutans cluster within family and racial cohorts: implications for natural transmission. 3216-3220 (1988).
38. Shaw, L. et al. The Human Salivary Microbiome Is Shaped by Shared Environment Rather than Genetics: Evidence from a Large Family of Closely Related Individuals. mBio 8, e01237-17 (2017).
39. McBain, A. J. Chapter 4: In vitro biofilm models: an overview. Adv. Appl. Microbiol. 69, 99-132 (2009).
40. Salli, K. M. & Ouwehand, A. C. The use of in vitro model systems to study dental biofilms associated with caries: a short review. J Oral Microbiol 7, 26149 (2015).
41. Guggenheim, B., Giertsen, E., Schüpbach, P. & Shapiro, S. Validation of an in vitro biofilm model of supragingival plaque. J. Dent. Res. 80, 363-370 (2001).
42. Schlafer, S. et al. pH Landscapes in a Novel Five-Species Model of Early Dental Biofilm. PLOS ONE 6, e25299 (2011).
43. Marsh, P. D. Dental plaque as a biofilm and a microbial community—implications for health and disease. BMC Oral Health 6, S14 (2006).
44. Floyd Dewhirst & Diane Hoffmann. A Case Study of a Hypothetical Oral Microbiome Transplant. in.
45. Allen-Vercoe, E. & Petrof, E. O. Artificial stool transplantation: progress towards a safer, more effective and acceptable alternative. Expert Review of Gastroenterology & Hepatology 7, 291-293 (2013).
46. Lee, C. H. et al. Frozen vs Fresh Fecal Microbiota Transplantation and Clinical Resolution of Diarrhea in Patients With Recurrent Clostridium difficile Infection: A Randomized Clinical Trial. JAMA 315, 142-149 (2016).
47. Cammarota, G., Ianiro, G. & Gasbarrini, A. Fecal microbiota transplantation for the treatment of Clostridium difficile infection: a systematic review. J. Clin. Gastroenterol. 48, 693-702 (2014).
48. Kassam, Z., Lee, C. H., Yuan, Y. & Hunt, R. H. Fecal microbiota transplantation for Clostridium difficile infection: systematic review and meta-analysis. Am. J. Gastroenterol. 108, 500-508 (2013).
49. van Nood, E. et al. Duodenal Infusion of Donor Feces for Recurrent Clostridium difficile. N Engl J Med 368, 407-415 (2013).
50. Costello, S. P. et al. Effect of Fecal Microbiota Transplantation on 8-Week Remission in Patients With Ulcerative Colitis: A Randomized Clinical Trial. JAMA 321, 156-164 (2019).
51. Dashper, S. G. et al. Casein Phosphopeptide-Amorphous Calcium Phosphate Reduces Streptococcus mutans Biofilm Development on Glass Ionomer Cement and Disrupts Established Biofilms. PLoS ONE 11, e0162322 (2016).
52. Bowen, W. H. Rodent model in caries research. Odontology 101, 9-14 (2013).
53. Keyes, P. H. Dental Caries in the Molar Teeth of Rats: I. Distribution of Lesions Induced by High-Carbohydrate Low-Fat Diets. J Dent Res 37, 1077-1087 (1958).
54. Symposium and Workshop on Animal Models in Cariology, Tanzer, J. M., & National Caries Program (U.S.). Animal models in cariology: proceedings of a Symposium and Workshop on Animal Models in Cariology, Apr. 21-23, 1980, Sheraton Sturbridge Inn, Sturbridge, Massachusetts. in (Information Retrieval, 1981).
55. Merits and modifications of scoring rat dental caries by Keyes”method|Scinapse|Academic search engine for paper. Scinapse https://scinapse.io/papers/117177179.
56. Weyrich, L. S. et al. Neanderthal behavior, diet, and disease inferred from ancient DNA in dental calculus. Nature 544, 357-361 (2017).
57. Thompson, L. R. et al. A communal catalogue reveals Earth's multiscale microbial diversity. Nature 551, 457-463 (2017).
58. Caporaso, J. G. et al. Ultra-high-throughput microbial community analysis on the Illumina HiSeq and MiSeq platforms. ISME J 6, 1621-1624 (2012).
59. Knapp, M., Stiller, M. & Meyer, M. Generating barcoded libraries for multiplex high-throughput sequencing. Methods Mol. Biol. 840, 155-170 (2012).
60. Meyer, M. & Kircher, M. Illumina sequencing library preparation for highly multiplexed target capture and sequencing. Cold Spring Harb Protoc 2010, pdb prot5448 (2010).
61. Bolyen, E. et al. QIHME 2: Reproducible, interactive, scalable, and extensible microbiome data science. https://peerj.com/preprints/27295 (2018) doi:10.7287/peerj.preprints.27295v1.
62. Davis, N. M., Proctor, D. M., Holmes, S. P., Relman, D. A. & Callahan, B. J. Simple statistical identification and removal of contaminant sequences in marker-gene and metagenomics data. Microbiome 6, 226 (2018).
63. Bolyen, E. et al. Reproducible, interactive, scalable and extensible microbiome data science using QIIME 2. Nature Biotechnology 37, 852-857 (2019).
64. Gonzalez, A. et al. Qiita: rapid, web-enabled microbiome meta-analysis. Nature Methods 15, 796 (2018).
65. Langille, M. G. I. et al. Predictive functional profiling of microbial communities using 16S rRNA marker gene sequences. Nat Biotech 31, 814-821 (2013).
66. Imelfort, M. et al. GroupM: an automated tool for the recovery of population genomes from related metagenomes. PeerJ 2, e603 (2014).
67. Beghini, F. et al. Integrating taxonomic, functional, and strain-level profiling of diverse microbial communities with bioBakery 3. eLife 10, e65088 (2021).
68. Bibbo, S. et al. Fecal Microbiota Transplantation: Screening and Selection to Choose the Optimal Donor. JClinMed 9, 1757 (2020).
69. Schueller, K., Riva, A., Pfeiffer, S., Berry, D. & Somoza, V. Members of the Oral Microbiota Are Associated with IL-8 Release by Gingival Epithelial Cells in Healthy Individuals. Front. Microbiol. 8, (2017).
70. Pekovid, D. D. & Fillery, E. D. Identification of bacteria in immunopathological mechanisms of human periodontal diseases. Journal of Periodontal Research 19, 329-351 (1984).

The present application is not to be limited in scope by the specific embodiments described herein. Indeed, various modifications of the embodiments in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

What is claimed is:

1. A method for treating dental caries or a periodontal disease, the method comprising:
a) collecting dental plaque from a donor;
b) growing a biofilm in vitro in a growth medium to modulate the oral microbial composition of the collected dental plaque from step a); and
c) transplanting the biofilm into an oral cavity of a recipient.

2. The method of claim 1, wherein the growth medium is artificial saliva (ASM) or SHI medium.

3. The method of claim 2, wherein the artificial saliva (ASM) comprises about 0.50 g/L tryptone, about 0.50 g/L neutralized bacteriological peptone, about 0.625 g/L type III porcine gastric mucin, about 0.25 g/L yeast extract, about 0.05 g/L KCl, about 0.05 g/L CaCl₂), about 0.088 g/L NaCl, and 1 mg/L haemin, or wherein SHI medium comprises about 5.0 g/L tryptone, about 10.0 g/L peptone, about 2.5 g/L type III porcine mucin, about 5.0 g/L yeast extract, about 1.0 mg/L Vitamin K (Sigma-Aldrich), about 2.5 g/L KCl, about 5.0 mg/L haemin, about 0.174 g/L L-arginine, about 0.06 g/L urea, about 5% v/v sheep blood, and about 10.0 mg/L N-acetylmuramic acid.

4. The method of claim 1, further comprising the following steps prior to step a):
1) providing a flow cell and at least one hydroxyapatite (HA) disc;
2) sterilizing the flow cell;
3) filling the flow cell with the growth medium devoid of sucrose;
4) mixing the dental plaque with a phosphate buffer solution (PBS) to form a first mixture;
5) adding the mixture to the growth medium to form a second mixture;
6) inoculating the second mixture into the flow cell;
7) flowing the flow cell with the growth medium; and
8) growing the biofilm on the HA disc for at least 10 days at about 36° C.

5. The method of claim 1, wherein at least 60% of the cells in the biofilm are viable.

6. The method of claim 1, wherein the biofilm comprises at least 200 bacterial species.

7. The method of claim 1, wherein the biofilm comprises aerobic and anaerobic bacteria.

8. The method of claim 7, wherein the anaerobic bacteria comprise Tannerella forsythia, Porphyromonas gingivalis, or a combination thereof.

9. The method of claim 1, wherein the microbiome of the biofilm has a different diversity than the microbiome of the collected dental plaque in step (a).

10. The method of claim 9, wherein the microbiome of the biofilm has a reduced diversity of the bacterial species by about 10% to about 22% relative to the collected dental plaque.

11. The method of claim 1, wherein the microbiome of the biofilm has reduced abundance in Actinobacteria, Fusobacteria, or a combination thereof relative to the microbiome of the collected dental plaque.

12. The method of claim 1, wherein the microbiome of the biofilm has increased abundance in Bacteriodetes, Firmicutes, and a combination thereof relative to the microbiome of the collected dental plaque, and/or wherein the microbiome of the biofilm has increased abundance in Bacilli spp., Haemophiluspara influenzae, or and a combination thereof relative to the microbiome of the collected dental plaque.

13. The method of claim 1, wherein the microbiome of the biofilm has reduced abundance of at least one oral pathogen.

14. The method of claim 13, where the oral pathogen comprises Porphyromonas gingivalis, Treponema denticola, or Tannerella forsythia, or any of the combinations thereof.

15. The method of claim 1, the biofilm reduces the proliferation of acidogenic and aciduric species or the proliferation of obligate anaerobic species.

16. The method of claim 1, the biofilm reduces the proliferation of Streptococcus mutans.

17. The method of claim 1, wherein the thickness of the biofilm is in the range of about 25 μm to about 55 μm, and/or wherein the biovolume of the biofilm is in the range of about 4 μm³ to about 8 μm³.

18. A method for modulating the oral microbial composition of a recipient, comprising:
a) collecting dental plaque from a donor;
b) culturing a biofilm in vitro in a medium to modulate the oral microbial composition of the collected dental plaque from step a); and
c) transplanting the biofilm into an oral cavity of a recipient.

19. A modulated oral microbial composition prepared by:
a) collecting dental plaque from a donor; and
b) growing a biofilm in vitro in a growth medium to modulate the oral microbial composition of the collected dental plaque from step a).

20. A system comprising a flow cell and at least one hydroxyapatite (HA) disc for use to modulate an oral microbial composition according to claim 1.
